SEQUENCE LISTING (14 sequences)

SEQ ID NO: 1 - protein, 14 aa, Cannabis sativa
NWIVAIKALIVIHR

SEQ ID NO: 2 - protein, 17 aa, Cannabis sativa
ILNLYSLASGQVINFAK

SEQ ID NO: 3 - protein, 11 aa, Cannabis sativa
LNTNSPNGFRR

SEQ ID NO: 4 - protein, 11 aa, Cannabis sativa
SVADLMYRSTR

SEQ ID NO: 5 - protein, 13 aa, Cannabis sativa
AVNTVILASDDMR

SEQ ID NO: 6 - protein, 27 aa, Cannabis sativa
VECEGGMIESWNPNHEQFQCAGVALLR

SEQ ID NO: 7 - protein, 16 aa, Cannabis sativa
AGIMDNGPNVTGPNEK

SEQ ID NO: 8 - protein, 12 aa, Cannabis sativa
VKEVLGGIWRLK

SEQ ID NO: 9 - protein, 9 aa, Cannabis sativa
KAIMVIRLK

SEQ ID NO: 10 - protein, 7 aa, Cannabis sativa
RMSCNFR

SEQ ID NO: 11 - protein, 11 aa, Cannabis sativa
SVADLMYRSTR

SEQ ID NO: 12 - protein, 16 aa, Cannabis sativa
QALLTFKQGLVDNQNR

SEQ ID NO: 13 - protein, 19 aa, Cannabis sativa
GFSVNLIQEAFNVDSETAR

SEQ ID NO: 14 - protein, 22 aa, Cannabis sativa
QRLQRCQQTCSQSQQGGQEQER

(19) United States
(12) Patent Application Publication
(10) Pub. No.: US 2024/0216296 A1
(43) Pub. Date: Jul. 4, 2024

(54) POLYPHENOL COMPOSITIONS HAVING IMPROVED BIOAVAILABILITY

(71) Applicant: EPICEUTICAL LABS LLC, Boulder, CO (US)

(72) Inventor: John GILDEA, Boulder, CO (US)

(21) Appl. No.: 18/558,124

(22) PCT Filed: Apr. 29, 2022
(86) PCT No.: PCT/US22/27115

§ 371 Date: Oct. 30, 2023

Related U.S. Application Data
(60) Provisional application No. 63/182,721, filed on Apr. 30, 2021.

(51) Int. Cl. A61K 31/12; A61K 9/00; A61K 31/05; A61K 31/353; A61K 31/4375; A61K 47/61; A61K 47/64; A61P 29/00

(52) U.S. Cl. CPC A61K 31/12; A61K 9/0053; A61K 31/05; A61K 31/353; A61K 31/4375; A61K 47/61; A61K 47/64; A61P 29/00

(57) ABSTRACT

Compositions and tri-molecular complexes of polyphenol-polysaccharide-protein are provided wherein the polyphenol is non-covalently complexed with covalently-linked polysaccharide and protein. The bioavailability of the polyphenol is such compositions is significantly increased in the disclosed compositions and tri-molecular complexes. Such compositions may be used to deliver high concentrations of the polyphenol for treating various conditions and diseases.

### FIELD

The present disclosure generally relates to the dietary supplements comprising a polyphenol, a polysaccharide, and a protein. More specifically, the present disclosure relates to dietary composition compositions that display significantly improved bioavailability as compared to a polyphenol itself.

### REFERENCE TO SEQUENCE LISTING

This application contains a sequence listing submitted as an electronic text file named “Sequence_Listing_10474-2-PCT_ST25.txt” having a size of 4,000 bytes and created on 29 Apr. 2022. The information contained in this electronic file is hereby incorporated by reference in its entirety pursuant to 37 C.F.R. § 1.52(e)(5).

### BACKGROUND

Polyphenols are naturally occurring antioxidant compounds found mainly in fruits, vegetables, cereals, and beverages of plant origin. Polyphenols are secondary metabolites of plants and are involved in defense against stressors such as ultraviolet (UV) radiation and infection, as well as being involved in cell division and hormonal regulation. All polyphenols are characterized by the presence of one or more phenolic groups, which can reduce reactive species and various organic substrates and minerals. These compounds may be classified into different groups as a function of the number of phenol rings they contain and based on the structural elements binding the rings to one another.

Over the last few decades, interest in polyphenols has grown. The main reason for this is the recognition of the antioxidant properties of polyphenols, their abundance in the human diet, and their role in preventing various diseases associated with oxidative stress, such as cancer, cardiovascular disease, and neurodegenerative diseases. In addition, polyphenols are known to modulate the activity of a wide range of enzymes and cell receptors. Thus, polyphenols are the subject of increasing scientific interest due to their possible beneficial effects on human health.

Certain polyphenols, such as quercetin, are found in all plant products, while others such as curcumin are specific to particular foods or plant species. In most cases, foods contain a mixture of polyphenols. To address this issue of variability, many companies produce supplements in which one or more polyphenols have been purified and/or concentrated, thereby allowing a consumer to easily ingest a quantity of a particular polyphenol. However, in many cases, the polyphenol has been purified from a plant, leaving the polyphenol in its native form. This can be problematic since most polyphenols are present in food in the form of esters, glycosides, or polymers, which are poorly absorbed. Thus, the polyphenols in many supplements have low bioavailability, meaning a large portion of the ingested polyphenol never reaches the bloodstream. Instead, it is destroyed in the stomach or excreted through the intestine. This results in the need to take larger and/or more frequent doses of a supplement to get a desired level of polyphenol in the bloodstream. The need for larger or increased number of doses results in wastage of material, as well as increased cost and inconvenience to the consumer.

### SUMMARY

Consequently, there is a need to for polyphenol compositions that enhance the bioavailability of dietary polyphenols. The present disclosure provides such compositions and related benefits as well.

In some embodiments the disclosure provides a composition with a polyphenol, a protein, and a polysaccharide, with the polysaccharide bound to the protein by a covalent bond between a carboxyl group on the polysaccharide and an amine group on the protein and the polyphenol non-covalently complexed with the covalently bound polysaccharide and protein, and the bioavailability of the polyphenol in the composition is greater than the bioavailability of the polyphenol in a composition lacking the covalently bound polysaccharide and protein.

In some embodiments the disclosure provides a method of making the preceding combination by coating the polysaccharide with the protein via transacylation reactions, by increasing the pH of the protein causing amine groups in the protein to lose a hydrogen atom, contacting the amine groups with carboxylic acids in the polysaccharide, forming a covalent bond between the amine groups and the carboxylic acids, and contacting the covalently bound protein and polysaccharide with the polyphenol, creating non-covalent interactions between the covalently bound protein and polysaccharide and the polyphenol.

In some embodiments the disclosure provides a composition with curcumin, alginate and hemp protein, with the alginate and hemp protein covalently bound to each other by a covalent bond between a carboxyl group on the alginate and an amine group on the hemp protein and the curcumin non-covalently complexed with the covalently bound alginate and hemp protein, and the bioavailability of the curcumin in the composition is greater than the bioavailability of the curcumin in a composition lacking the covalently bound alginate and hemp protein.

In some embodiments the disclosure provides a method of making the preceding combination by coating the alginate with the hemp protein via transacylation reactions, by increasing the pH of the hemp protein causing amine groups in the hemp protein to lose a hydrogen atom, contacting the amine groups with carboxylic acids in the alginate, forming a covalent bond between the amine groups and the carboxylic acids, and contacting the covalently bound alginate and hemp protein with the curcumin, creating non-covalent interactions between the covalently bound alginate and hemp protein and the curcumin.

In some embodiments the disclosure provides a composition with berberine, propylene glycol alginate and pea protein, with the propylene glycol alginate and pea protein covalently bound to each other by a covalent bond between a carboxyl group on the propylene glycol alginate and an amine group on the pea protein and the berberine non-covalently complexed with the covalently bound propylene glycol alginate and pea protein, and the bioavailability of the berberine in the composition is greater than the bioavailability of the berberine in a composition lacking the covalently bound propylene glycol alginate and pea protein.

In some embodiments the disclosure provides a method of making the preceding combination by coating the propylene glycol alginate with the pea protein via transacylation reactions by increasing the pH of the pea protein causing amine groups in the pea protein to lose a hydrogen atom, contacting the amine groups with carboxylic acids in the propylene glycol alginate, forming a covalent bond between the amine groups and the carboxylic acids, and contacting the covalently bound propylene glycol alginate and pea protein with the quercetin, creating non-covalent interactions between the covalently bound propylene glycol alginate and pea protein and the quercetin.

In some embodiments the disclosure provides a composition with resveratrol, propylene glycol alginate and hemp protein, with the propylene glycol alginate and hemp protein covalently bound to each other by a covalent bond between a carboxyl group on the propylene glycol alginate and an amine group on the hemp protein and the resveratrol non-covalently complexed with the covalently bound propylene glycol alginate and hemp protein, and the bioavailability of the resveratrol in the composition is greater than the bioavailability of the resveratrol in a composition lacking the covalently bound propylene glycol alginate and hemp protein.

In some embodiments the disclosure provides a method of making the preceding combination by coating the propylene glycol alginate with the hemp protein via transacylation reactions, by increasing the pH of the hemp protein causing amine groups in the hemp protein to lose a hydrogen atom, contacting the amine groups with carboxylic acids in the propylene glycol alginate, forming a covalent bond between the amine groups and the carboxylic acids, and contacting the covalently bound propylene glycol alginate and hemp protein with the resveratrol, creating non-covalent interactions between the covalently bound propylene glycol alginate and hemp protein and the resveratrol.

### BRIEF DESCRIPTION OF THE FIGURES

FIG. 1 shows urinary concentrations of curcumin after ingestion of various formulations containing curcumin. Abbreviations are as described in Example 2.

FIG. 2 shows urine curcumin levels as described in Example 4.

FIG. 3 shows urine IL6 levels as described in Example 4.

FIGS. 4A, 4B, 4C, 4D, 4E, 4F, 4G, 4H, and 4I show hydropathy plots for various polypeptides, as described in Example 6.

### DETAILED DESCRIPTION

Compositions and methods of making such compositions are provided herein that address the afore-mentioned problems. In particular, compositions comprising a polyphenol, a polysaccharide, for example an alginate or other fiber, and a protein are provided. The disclosed compositions display significantly increased polyphenol bioavailability. In various aspects, complexes comprising a polyphenol, a polysaccharide which in some embodiments is an alginate, and a protein are provided. Providing a polyphenol to a subject in such a tri-molecular complex greatly increases the bioavailability of the polyphenol in the subject. Moreover, such compositions may be used to treat conditions and diseases/disorders that are responsive to polyphenol therapy.

The trimolecular complex increases bioavailability of polyphenols, at least in part by increasing solubility. This is accomplished by at least in part surrounding a polyphenol, which can be hydrophobic, with one or more proteins. The one or more proteins provide a hydrophilic exterior, providing amphipathic characteristics to the complex, thereby increasing solubility of the polyphenol.

In various aspects, the trimolecular complex contains a polysaccharide that can be a fiber, which can be an indigestible fiber, a digestible fiber, a prebiotic fiber, and/or a different type of fiber. In some embodiments, the fiber is a dietary fiber.

In some embodiments, the trimolecular complex contains an indigestible fiber. In some embodiments, the trimolecular complex contains a polysaccharide, which can be an indigestible fiber. An indigestible fiber can be used to slow transit of the trimolecular complex and/or the polyphenol through the small intestine. A muco-adherent and/or mucin binding fiber and/or polysaccharide can slow transit of the trimolecular complex and/or the polyphenol through the small intestine. Examples of indigestible muco-adherent and/or mucin binding fibers are fructooligosacharride (FOS), alginate, and pectin.

In some embodiments, the trimolecular complex contains a prebiotic fiber. A prebiotic fiber can expedite transit through the small intestine to the colon and can be digested in the colon and released. For example, galactooligosaccharides (GOS) or resistant starch, e.g., potato starch, are prebiotic fibers that are digested in the colon and released. In this instance, the fiber does not adhere to the mucus layer and is released in the colon via biotransformation by the colonic microbiome.

Disclosed herein are methods of increasing solubility and/or bioavailability of a polyphenol by non-covalent attachment of the polyphenol to a protein. Without being bound by theory, one or more hydrophobic portions of a protein bind(s) non-covalently to a polyphenol, thereby increasing hydrophilicity of the polyphenol by forming a protein-polyphenol complex. This increases solubility and/or bioavailability of the polyphenol.

Solubility and bioavailability can be determined by any number of means. For example, bioavailability can be determined by high-performance liquid chromatography with fluorescence detection (HPLC-FL) using the following chromatographic conditions: Luna column (C18; 150 4 mm; 3 mm), acetonitrile:acetic acid pH 3.2 (45:55 to 60:40) as mobile phase, flow rate of 1 mL per minute, excitation at 429/285 nm and emission at 529 nm and injection of 10 mL. Plasma samples can be extracted using ethyl acetate and methanol (95:5, 500 mL) and estradiol (30 mg mL 1) as internal standard, with subsequent stirring (3 min) and centrifugation (8 min) (triple extraction). The organic fraction can be evaporated under N2 (20 min) and the dried residue reconstituted in acetonitrile.

In some embodiments, the trimolecular complex causes an effect determined by the identity of the polyphenol. For example, curcumin-containing trimolecular complexes can cause decreases in inflammatory markers and inflammation. A curcumin-containing trimolecular complex can cause decreased inflammation, as determined by blood and/or urine levels of IL6. Without being bound by theory, curcumin can inhibit NFκB inflammatory signaling. IL6 is a secreted inflammatory cytokine encoded by an NFκB-regulated gene. IL6 is secreted into the blood and filtered by the kidney and removed from the body through the urine. It is a marker of inflammation going on anywhere in the body.

As used herein, the singular forms “a,” “an,” and “the” include plural referents unless the context clearly dictates otherwise. For example, a polyphenol refers to one or more polyphenols. As such, the terms “a.” “an,” “one or more,” and “at least one” can be used interchangeably. Similarly, the terms “comprising,” “including,” and “having” can be used interchangeably.

As used herein, unless otherwise specified, the terms “about,” “approximately,” and the like, when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” and the like mean that each of the limitations or ranges may vary by up to about 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

Certain features of the present disclosure are described in the context of separate embodiments but may also be provided in combination in a single embodiment. Conversely, various features of the present disclosure, which are described in the context of a single embodiment may also be provided separately or in any suitable sub-combination(s). All combinations of the disclosed embodiments are specifically embraced by the present disclosure and are disclosed herein as if each and every combination was individually and explicitly disclosed. In addition, all sub-combinations are also specifically embraced by the present disclosure and are disclosed herein as if each and every such sub-combination was individually and explicitly disclosed.

The present disclosure is not limited to particular embodiments described herein. Terminology used herein for describing particular embodiments only is not intended to be limiting. Amy publications discussed herein are provided solely for their disclosure prior to the filing date of the present application. Nothing herein is an admission that the present disclosure is not entitled to antedate such publication by virtue of prior disclosure. Further, the publication dates provided may be different from actual publication dates, which may need to be independently confirmed.

Although any methods and materials similar or equivalent to those described herein can be used in the practice or testing of the methods and compositions disclosed herein, the preferred methods and materials are now described.

### Compositions

In various aspects, the present disclosure provides compositions comprising, consisting of, or consisting essentially of, a complex of a polyphenol, a polysaccharide, and a protein, wherein the polysaccharide is covalently bound to the protein. For ease of discussion, such complex is referred to throughout this disclosure as a tri-molecular complex. In the disclosed compositions, the bioavailability of the polyphenol in the disclosed tri-molecular complexes is significantly greater relative to the bioavailability of the polyphenol by itself and/or in another composition, for example a composition lacking the polysaccharide and/or the protein, or in which the polysaccharide is not covalently bound to the protein. As used herein, the term “bioavailability” generally means the rate and extent (e.g., as measured in percent) to which a compound such as a polyphenol is absorbed from an administered composition or complex, reaches the systemic circulation and becomes available at one or more desired sites of action. For oral dosage forms, bioavailability relates to the processes by which the active ingredient is released from the oral dosage form, absorbed through the stomach or intestinal wall, and moves into the systemic circulation and to a desired site of action. Bioavailability data for a particular composition/formulation provides an estimate of the fraction of the administered dose, for example that portion of an active ingredient formulated in an oral tablet or capsule, that is absorbed into the systemic circulation.

Bioavailability can be measured in blood, urine, or other body fluids or tissues. For example, a formulation can be administered to an adult subject, and blood, urine, or other body fluid or tissue samples can be collected. For example, a sample from the morning void of urine can be taken as a control. Following control sample collection, each subject can be administered the formulation, for example 1.8 grams (g) of the formulation. After 4 hours, a second urine sample can be taken, spun, and the amount of polyphenol, for example curcumin, quercetin, berberine, or resveratrol present in the urine sample can determined by fluorescence. Briefly, the sample can be subjected to light at a frequency of 430 nM, and emission measured at 530 nM. The resulting emissions can then be used to quantify the amount of polyphenol in the sample, as is commonly known in the art.

In various aspects, the polysaccharide and the protein in the tri-molecular complex are bound by a covalent bond. Formation of the covalent bond can occur via trans-acylation. In some embodiments, the covalent bond is between a carboxyl group and an amine group. In some embodiments, the polysaccharide is bound to the protein by a covalent bond between a carboxyl group on the polysaccharide and an amine group on the protein.

### Polyphenols

As used herein, “polyphenol” refers to a family of mainly natural, but also synthetic or semi-synthetic, organic molecules characterized by the presence of multiple phenolic units. The number and structure of the phenol units in a particular polyphenol molecules give rise, at least in part, to the unique physical, chemical, and biological properties of the polyphenol.

Polyphenols may be divided into four classes based on structure: flavonoids, phenolic acids, stilbenes, and lignans, though there are other polyphenols, for example the curcuminoids, that do not easily fit within any of these four classes. Flavonoids are characterized by the presence of a flavone backbone structure, which is a 15-carbon skeleton containing two phenyl rings and a heterocyclic ring containing an embedded oxygen:

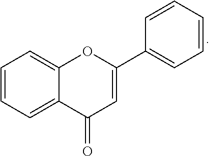 [SMILES: O=c1cc(-c2ccccc2)oc2ccccc12]

The flavone carbon structure is typically abbreviated as C6-C3-C6. Phenolic acids, or phenolcarboxylic acids, are aromatic compounds and include substances containing a phenolic ring and an organic carboxylic acid function (C6-C1 skeleton) A stilbene is a diarylethene possessing a central ethylene moiety with one phenyl group substituent on each end of the carbon-carbon double bond. Lignans are low molecular weight polyphenols that possess a C18 core. In one aspect, the polyphenol is a flavonoid. In one aspect, the polyphenol is a phenolic acid. In one aspect, the polyphenol is a stilbene. In one aspect, the polyphenol is a lignan.

In various aspects provided by the present disclosure, the polyphenol in a composition or complex provided by the disclosure is an active agent, ingredient or component in that it possesses one or more biological activities. As used herein, the phrase “biological activity,” and like phrases, refers to the ability of a polyphenol to affect, or modulate, one or more biological systems or biological molecules, such as a protein or enzyme. As such, the polyphenol may be referred to as an active agent in a composition or complex. Examples of such biological activity include, but are not limited to, anti-inflammatory activity, antioxidant activity, alteration of cellular redox potential, alteration of enzymatic activity, antibacterial activity, inhibition of cell proliferation, regulation of nuclear transcription factors, regulation of fat metabolism, modulation of inflammatory mediators, including tumor necrosis factor α (TNF-α), interleukin (IL)-1β, and IL6, modulation of insulin secretion, reduction off apoptosis, promotion of β-cell proliferation, reduction of insulin resistance, inhibition of sodium glucose cotransporter type-1 (SGLT-1) and/or sodium glucose cotransporter type-2 (SGLT-2), reduction of blood pressure, improvement of endothelial function, reduction of C-reactive protein, reducing the risk of cardiovascular disease, modulating nitric oxide (NO) production, reducing low-density lipid (LDL) cholesterol, reducing systolic and/or diastolic blood pressure, reducing fasting glucose, reducing HbA1c, reducing body mass index, activating sirtuins, improving peripheral blood flow, reducing cognitive decline, reducing the risk for depression, improving attention and executive function, increasing brain-derived neurotrophic factor (BDNF), and protecting against Parkinson's disease, among other activities.

In certain aspects, the polyphenol in a composition or complex provided by the disclosure may affect one or more biochemical pathways. According to the present disclosure, a polyphenol that affects a biochemical pathway can modulate one or more molecules (e.g., enzymes, substrates, products, cofactors, etc.) in a biochemical pathway. Modulating a molecule may refer to increasing or decreasing the level or activity of a molecule. Examples of such biochemical pathways include, but are not limited to, the mechanistic target of rapamycin (mTOR) pathway, the Sirtuin 1 (SIRT1) pathway, a peroxisome proliferator-activated receptor-gamma coactivator (PGC)-1alpha pathway, the autophagy/proteostasis pathway, an AMP-activated protein kinase (AMPK) pathway, a c-Myc pathway, a nuclear factor-κB (NF-κB) pathway, a nuclear factor erythroid 2-related factor 2 (Nrf2) pathway, a forkhead box O-3 (FoxO3) pathway, an uncoupling protein 1 (UCP-1) pathway, a signal transduction pathway, and a pathway involved in the clearance of senescent cells. In one aspect, the polyphenol inhibits NF-κB. In one aspect, the polyphenol induces the NrF2 pathway. In one aspect, the polyphenol inhibits mTOR signaling. In one aspect, the polyphenol induces apoptosis and/or autophagy.

In some embodiments, the polyphenol is anti-inflammatory capable of reducing inflammation. In some embodiments, the polyphenol is an antioxidant capable of reducing the presence of free radicals. In some embodiments, the polyphenol is neuroprotective. In some embodiments, the polyphenol affects cell growth. In other embodiments, the polyphenol inhibits cell growth. In those embodiments in which the disclosed polyphenol impacts cell growth, the cell may be in vitro or in vivo. In some embodiments, the polyphenol may alter the oxidation-reduction status of a cell. In some embodiments, the polyphenol affects the expression of a gene.

Examples of polyphenols suitable for inclusion in compositions of the disclosure are disclosed herein and may also be found, for example, in U.S. Patent Publication No. 2018/0140709, U.S. Pat. No. 10,968,260, and/or U.S. Pat. No. 10,898,477. In one embodiment, the polyphenol in a complex and/or composition provided by the present disclosure is selected from the group consisting of a turmeric extract, curcuminoids, curcumin, quercetin, resveratrol, 6-shogaol, fisetin, naringin, apigenin, pterostilbene, baicalin, berberine, silibinin (a.k.a., silybin), Silymarin, ursolic acid, xanthohumol, Boswellia, catechin, epigallocatechin gallate (EGCG), enterodiol, enterolactone, and withanolides. In another embodiment, the polyphenol in a complex and/or composition provided by the present disclosure is selected from the group consisting of resveratrol, berberine, curcumin, quercetin, EGCG, silymarin, and ursolic acid. In one aspect, polyphenols present in a composition or complex provided by the present disclosure are selected from the polyphenols listed in Table 1, inclusive of combinations thereof:

TABLE 1
Polyphenols

| | |
|---|---|
| Cyanidin | Quercetin 3-O-xylosyl-rutinoside |
| Cyanidin 3,5-O-diglucoside | Quercetin 4′-O-glucoside |
| Cyanidin 3-O-(6″-acetyl-galactoside) | Quercetin 7,4′-O-diglucoside |
| Cyanidin 3-O-(6″-acetyl-glucoside) | Rhamnetin |
| Cyanidin 3-O-(6″-caffeoyl-glucoside) | Spinacetin 3-O-(2″-feruloylglucosyl)(1->6)- |
| | [apiosyl(1->2)]-glucoside |
| Cyanidin 3-O-(6″-dioxalyl-glucoside) | Spinacetin 3-O-(2″-p-coumaroylglucosyl)(1->6)- |
| | [apiosyl(1->2)]-glucoside |
| Cyanidin 3-O-(6″-malonyl-3″-glucosyl- | Spinacetin 3-O-glucosyl-(1->6)-glucoside |
| glucoside) | |
| Cyanidin 3-O-(6″-malonyl-glucoside) | Spinacetin 3-O-glucosyl-(1->6)- |
| | [apiosyl(1->2)]-glucoside |
| Cyanidin 3-O-(6″-p-coumaroyl-glucoside) | 6″-O-Acetyldaidzin |
| Cyanidin 3-O-(6″-succinyl-glucoside) | 6″-O-Acetylgenistin |
| Cyanidin 3-O-arabinoside | 6″-O-Acetylglycitin |
| Cyanidin 3-O-galactoside | 6″-O-Malonyldaidzin |
| Cyanidin 3-O-glucoside | 6″-O-Malonylgenistin |
| Cyanidin 3-O-glucosyl-rutinoside | 6″-O-Malonylglycitin |
| Cyanidin 3-O-rutinoside | Biochanin A |
| Cyanidin 3-O-sambubioside | Daidzein |
| Cyanidin 3-O-sambubioside 5-O-glucoside | Daidzin |
| Cyanidin 3-O-sophoroside | Formononetin |
| Cyanidin 3-O-xyloside | Genistein |
| Cyanidin 3-O-xylosyl-rutinoside | Genistin |
| Delphinidin 3,5-O-diglucoside | Glycitein |
| Delphinidin 3-O-(6″-acetyl-galactoside) | Glycitin |
| Delphinidin 3-O-(6″-acetyl-glucoside) | 1-Acetoxypinoresinol |
| Delphinidin 3-O-(6″-p-coumaroyl-glucoside) | 7-Hydroxymatairesinol |
| Delphinidin 3-O-arabinoside | 7-Hydroxysecoisolariciresinol |
| Delphinidin 3-O-feruloyl-glucoside | 7-Oxomatairesinol |
| Delphinidin 3-O-galactoside | Anhydro-secoisolariciresinol |
| Delphinidin 3-O-glucoside | Arctigenin |
| Delphinidin 3-O-glucosyl-glucoside | Conidendrin |
| Delphinidin 3-O-rutinoside | Cyclolariciresinol |
| Delphinidin 3-O-sambubioside | Dimethylmatairesinol |
| Delphinidin 3-O-xyloside | Episesamin |
| Malvidin 3,5-O-diglucoside | Episesaminol |
| Malvidin 3-O-(6″-acetyl-galactoside) | Isohydroxymatairesinol |
| Malvidin 3-O-(6″-acetyl-glucoside) | Isolariciresinol |
| Malvidin 3-O-(6″-caffeoyl-glucoside) | Lariciresinol |
| Malvidin 3-O-(6″-p-coumaroyl-glucoside) | Lariciresinol-sesquilignan |
| Malvidin 3-O-arabinoside | Matairesinol |
| Malvidin 3-O-galactoside | Medioresinol |
| Malvidin 3-O-glucoside | Nortrachelogenin |
| Pelargonidin | Pinoresinol |
| Pelargonidin 3,5-O-diglucoside | Secoisolariciresinol |
| Pelargonidin 3-O-(6″-malonyl-glucoside) | Secoisolariciresinol-sesquilignan |
| Pelargonidin 3-O-(6″-succinyl-glucoside) | Sesamin |
| Pelargonidin 3-O-arabinoside | Sesaminol |
| Pelargonidin 3-O-galactoside | Sesamol |
| Pelargonidin 3-O-glucoside | Sesamolin |
| Pelargonidin 3-O-glucosyl-rutinoside | Sesamolinol |
| Pelargonidin 3-O-rutinoside | Syringaresinol |
| Pelargonidin 3-O-sambubioside | Todolactol A |
| Pelargonidin 3-O-sophoroside | Trachelogenin |
| Peonidin | 4-Ethylguaiacol |
| Peonidin 3-O-(6″-acetyl-galactoside) | 4-Vinylguaiacol |
| Peonidin 3-O-(6″-acetyl-glucoside) | 4-Vinylsyringol |
| Peonidin 3-O-(6″-p-coumaroyl-glucoside) | 3-Methylcatechol |
| Peonidin 3-O-arabinoside | 4-Ethylcatechol |
| Peonidin 3-O-galactoside | 4-Ethylphenol |
| Peonidin 3-O-glucoside | 4-Methylcatechol |
| Peonidin 3-O-rutinoside | 4-Vinylphenol |
| Petunidin 3,5-O-diglucoside | 5-Heneicosenylresorcinol |
| Petunidin 3-O-(6″-acetyl-galactoside) | 5-Heneicosylresorcinol |
| Petunidin 3-O-(6″-acetyl-glucoside) | 5-Heptadecylresorcinol |
| Petunidin 3-O-(6″-p-coumaroyl-glucoside) | 5-Nonadecenylresorcinol |
| Petunidin 3-O-arabinoside | 5-Nonadecylresorcinol |
| Petunidin 3-O-galactoside | 5-Pentacosenylresorcinol |
| Petunidin 3-O-glucoside | 5-Pentacosylresorcinol |
| Petunidin 3-O-rhamnoside | 5-Pentadecylresorcinol |
| Petunidin 3-O-rutinoside | 5-Tricosenylresorcinol |
| Pigment A | 5-Tricosylresorcinol |
| Pinotin A | Bisdemethoxycurcumin |
| Vitisin A | Curcumin |
| Butein | Demethoxycurcumin |
| Xanthohumol | Bergapten |
| 3-Hydroxyphloretin 2′-O-glucoside | Isopimpinellin |
| 3-Hydroxyphloretin 2′-O-xylosyl-glucoside | Psoralen |
| Phloretin | Xanthotoxin |
| Phloretin 2′-O-xylosyl-glucoside | 4-Hydroxybenzaldehyde |
| Phloridzin | Gallic aldehyde |
| Dihydromyricetin 3-O-rhamnoside | p-Anisaldehyde |
| Dihydroquercetin | Protocatechuic aldehyde |
| Dihydroquercetin 3-O-rhamnoside | Syringaldehyde |
| (+)-Catechin | Vanillin |
| (+)-Catechin 3-O-gallate | 2,3-Dihydroxy-1-guaiacylpropanone |
| (+)-Catechin 3-O-glucose | 3-Methoxyacetophenone |
| (+)-Gallocatechin | Ferulaldehyde |
| (+)-Gallocatechin 3-O-gallate | Sinapaldehyde |
| (−)-Epicatechin | 4-Hydroxycoumarin |
| (−)-Epicatechin 3-O-gallate | Coumarin |
| (−)-Epicatechin-(2a-7)(4a-8)-epicatechin 3- | Esculetin |
| O-galactoside | |
| (−)-Epigallocatechin | Esculin |
| (−)-Epigallocatechin 3-O-gallate | Mellein |
| Cinnamtannin A2 | Scopoletin |
| Procyanidin dimer B1 | Umbelliferone |
| Procyanidin dimer B2 | 2-Methoxy-5-prop-1-enylphenol |
| Procyanidin dimer B3 | Acetyl eugenol |
| Procyanidin dimer B4 | Anethole |
| Procyanidin dimer B5 | Estragole |
| Procyanidin dimer B7 | Eugenol |
| Procyanidin trimer C1 | [6]-Gingerol |
| Procyanidin trimer C2 | Guaiacol |
| Procyanidin trimer EEC | 1,4-Naphtoquinone |
| Procyanidin trimer T2 | Juglone |
| Prodelphinidin dimer B3 | 3,4-Dihydroxyphenylglycol |
| Prodelphinidin trimer C-GC-C | Arbutin |
| Prodelphinidin trimer GC-C-C | Catechol |
| Prodelphinidin trimer GC-GC-C | Coumestrol |
| Theaflavin | Phenol |
| Theaflavin 3′-O-gallate | Phlorin |
| Theaflavin 3,3′-O-digallate | Pyrogallol |
| Theaflavin 3-O-gallate | Carnosic acid |
| 6-Geranylnaringenin | Carnosol |
| 6-Prenylnaringenin | Carvacrol |
| 8-Prenylnaringenin | Epirosmanol |
| Didymin | Rosmadial |
| Eriocitrin | Rosmanol |
| Eriodictyol | Thymol |
| Eriodictyol 7-O-glucoside | 3,4-DHPEA-AC |
| Hesperetin | 3,4-DHPEA-EA |
| Hesperidin | 3,4-DHPEA-EDA |
| Isoxanthohumol | Demethyloleuropein |
| Naringenin | Hydroxytyrosol |
| Naringenin 7-O-glucoside | Hydroxytyrosol 4-O-glucoside |
| Naringin | Ligstroside |
| Naringin 4′-O-glucoside | Ligstroside-aglycone |
| Naringin 6′-malonate | Oleoside 11-methylester |
| Narirutin | Oleoside dimethylester |
| Narirutin 4′-O-glucoside | Oleuropein |
| Neoeriocitrin | Oleuropein-aglycone |
| Neohesperidin | p-HPEA-AC |
| Pinocembrin | p-HPEA-EA |
| Poncirin | p-HPEA-EDA |
| Sakuranetin | Tyrosol |
| 5,6-Dihydroxy-7,8,3′,4′-tetramethoxyflavone | 2,3-Dihydroxybenzoic acid |
| 6-Hydroxyluteolin | 2,4-Dihydroxybenzoic acid |
| 6-Hydroxyluteolin 7-O-rhamnoside | 2,6-Dihydroxybenzoic acid |
| 7,3′,4′-Trihydroxyflavone | 2-Hydroxybenzoic acid |
| 7,4′-Dihydroxyflavone | 3,5-Dihydroxybenzoic acid |
| Apigenin | 3-Hydroxybenzoic acid |
| Apigenin 6,8-C-arabinoside-C-glucoside | 4-Hydroxybenzoic acid |
| Apigenin 6,8-C-galactoside-C-arabinoside | 4-Hydroxybenzoic acid 4-O-glucoside |
| Apigenin 6,8-di-C-glucoside | 5-O-Galloylquinic acid |
| Apigenin 6-C-glucoside | Benzoic acid |
| Apigenin 7-O-(6″-malonyl-apiosyl- | Ellagic acid |
| glucoside) | |
| Apigenin 7-O-apiosyl-glucoside | Ellagic acid acetyl-arabinoside |
| Apigenin 7-O-diglucuronide | Ellagic acid acetyl-xyloside |
| Apigenin 7-O-glucoside | Ellagic acid arabinoside |
| Apigenin 7-O-glucuronide | Ellagic acid glucoside |
| Baicalein | Gallic acid |
| Chrysin | Gallic acid 3-O-gallate |
| Chrysoeriol 7-O-(6″-malonyl-apiosyl- | Gallic acid 4-O-glucoside |
| glucoside) | |
| Chrysoeriol 7-O-(6″-malonyl-glucoside) | Gallic acid ethyl ester |
| Chrysoeriol 7-O-apiosyl-glucoside | Galloyl glucose |
| Chrysoeriol 7-O-glucoside | Gentisic acid |
| Cirsilineol | Lambertianin C |
| Cirsimaritin | Protocatechuic acid |
| Diosmin | Protocatechuic acid 4-O-glucoside |
| Eupatorin | Punicalagin |
| Gardenin B | Sanguiin H-6 |
| Geraldone | Syringic acid |
| Hispidulin | Valoneic acid dilactone |
| Isorhoifolin | Vanillic acid |
| Jaceosidin | 1,2′-Disinapoyl-2-feruloylgentiobiose |
| Luteolin | 1,2,2′-Triferuloylgentiobiose |
| Luteolin 6-C-glucoside | 1,2,2′-Trisinapoylgentiobiose |
| Luteolin 7-O-(2-apiosyl-6-malonyl)- | 1,2-Diferuloylgentiobiose |
| glucoside | |
| Luteolin 7-O-(2-apiosyl-glucoside) | 1,2-Disinapoylgentiobiose |
| Luteolin 7-O-diglucuronide | 1-Sinapoyl-2,2′-diferuloylgentiobiose |
| Luteolin 7-O-glucoside | 1-Sinapoyl-2-feruloylgentiobiose |
| Luteolin 7-O-glucuronide | 2,5-di-S-Glutathionyl caftaric acid |
| Luteolin 7-O-malonyl-glucoside | 2-S-Glutathionyl caftaric acid |
| Luteolin 7-O-rutinoside | 24-Methylcholestanol ferulate |
| Neodiosmin | 24-Methylcholesterol ferulate |
| Nepetin | 24-Methylenecholestanol ferulate |
| Nobiletin | 24-Methyllathosterol ferulate |
| Pebrellin | 3,4-Dicaffeoylquinic acid |
| Rhoifolin | 3,4-Diferuloylquinic acid |
| Rhoifolin 4′-O-glucoside | 3,5-Dicaffeoylquinic acid |
| Scutellarein | 3,5-Diferuloylquinic acid |
| Sinensetin | 3-Caffeoylquinic acid |
| Tangeretin | 3-Feruloylquinic acid |
| Tetramethylscutellarein | 3-p-Coumaroylquinic acid |
| 3,7-Dimethylquercetin | 3-Sinapoylquinic acid |
| 3-Methoxynobiletin | 4,5-Dicaffeoylquinic acid |
| 3-Methoxysinensetin | 4-Caffeoylquinic acid |
| 5,3′,4′-Trihydroxy-3-methoxy-6:7- | 4-Feruloylquinic acid |
| methylenedioxyflavone 4′-O-glucuronide | |
| 5,4′-Dihydroxy-3,3′-dimethoxy-6:7- | 4-p-Coumaroylquinic acid |
| methylenedioxyflavone 4′-O-glucuronide | |
| 6,8-Dihydroxykaempferol | 4-Sinapoylquinic acid |
| Galangin | 5-5′-Dehydrodiferulic acid |
| Isorhamnetin | 5-8′-Benzofuran dehydrodiferulic acid |
| Isorhamnetin 3-O-galactoside | 5-8′-Dehydrodiferulic acid |
| Isorhamnetin 3-O-glucoside | 5-Caffeoylquinic acid |
| Isorhamnetin 3-O-glucoside 7-O-rhamnoside | 5-Feruloylquinic acid |
| Isorhamnetin 3-O-glucuronide | 5-p-Coumaroylquinic acid |
| Isorhamnetin 3-O-rutinoside | 5-Sinapoylquinic acid |
| Isorhamnetin 4′-O-glucoside | 8-O-4′-Dehydrodiferulic acid |
| Isorhamnetin 7-O-rhamnoside | Avenanthramide 2c |
| Jaceidin 4′-O-glucuronide | Avenanthramide 2f |
| Kaempferide | Avenanthramide 2p |
| Kaempferol | Avenanthramide K |
| Kaempferol 3,7,4′-O-triglucoside | Caffeic acid |
| Kaempferol 3,7-O-diglucoside | Caffeic acid 4-O-glucoside |
| Kaempferol 3-O-(2″-rhamnosyl-6″-acetyl- | Caffeic acid ethyl ester |
| galactoside) 7-O-rhamnoside | |
| Kaempferol 3-O-(2″-rhamnosyl-galactoside) | Caffeoyl aspartic acid |
| 7-O-rhamnoside | |
| Kaempferol 3-O-(6″-malonyl-glucoside) | Caffeoyl glucose |
| Kaempferol 3-O-(6″-acetyl-galactoside) 7-O- | Caffeoyl tartaric acid |
| rhamnoside | |
| Kaempferol 3-O-acetyl-glucoside | Chicoric acid |
| Kaempferol 3-O-galactoside | Cinnamic acid |
| Kaempferol 3-O-galactoside 7-O-rhamnoside | Cinnamoyl glucose |
| Kaempferol 3-O-glucoside | Ferulic acid |
| Kaempferol 3-O-glucosyl-rhamnosyl- | Ferulic acid 4-O-glucoside |
| galactoside | |
| Kaempferol 3-O-glucosyl-rhamnosyl- | Feruloyl glucose |
| glucoside | |
| Kaempferol 3-O-glucuronide | Feruloyl tartaric acid |
| Kaempferol 3-O-rhamnoside | Hydroxycaffeic acid |
| Kaempferol 3-O-rhamnosyl-rhamnosyl- | Isoferulic acid |
| glucoside | |
| Kaempferol 3-O-rutinoside | m-Coumaric acid |
| Kaempferol 3-O-sophoroside | o-Coumaric acid |
| Kaempferol 3-O-sophoroside 7-O-glucoside | p-Coumaric acid |
| Kaempferol 3-O-xylosyl-glucoside | p-Coumaric acid 4-O-glucoside |
| Kaempferol 3-O-xylosyl-rutinoside | p-Coumaric acid ethyl ester |
| Kaempferol 7-O-glucoside | p-Coumaroyl glucose |
| Methylgalangin | p-Coumaroyl glycolic acid |
| Morin | p-Coumaroyl malic acid |
| Myricetin | p-Coumaroyl tartaric acid |
| Myricetin 3-O-arabinoside | p-Coumaroyl tartaric acid glucosidic ester |
| Myricetin 3-O-galactoside | p-Coumaroyl tyrosine |
| Myricetin 3-O-glucoside | p-Coumaroylquinic acid |
| Myricetin 3-O-rhamnoside | Rosmarinic acid |
| Myricetin 3-O-rutinoside | Schottenol ferulate |
| Patuletin 3-O-(2″-feruloylglucosyl)(1->6)- | Sinapic acid |
| [apiosyl(1->2)]-glucoside | |
| Patuletin 3-O-glucosyl-(1->6)- | Sinapine |
| [apiosyl(1->2)]-glucoside | |
| Quercetin | Sitosterol ferulate |
| Quercetin 3,4′-O-diglucoside | Stigmastanol ferulate |
| Quercetin 3-O-(6″-malonyl-glucoside) | Verbascoside |
| Quercetin 3-O-(6″-malonyl-glucoside) 7-O- | 3,4-Dihydroxyphenylacetic acid |
| glucoside | |
| Quercetin 3-O-(6″-acetyl-galactoside) 7-O- | 4-Hydroxyphenylacetic acid |
| rhamnoside | |
| Quercetin 3-O-acetyl-rhamnoside | Homovanillic acid |
| Quercetin 3-O-arabinoside | Homoveratric acid |
| Quercetin 3-O-galactoside | Methoxyphenylacetic acid |
| Quercetin 3-O-galactoside 7-O-rhamnoside | Dihydro-p-coumaric acid |
| Quercetin 3-O-glucoside | Dihydrocaffeic acid |
| Quercetin 3-O-glucosyl-rhamnosyl- | d-Viniferin |
| galactoside | |
| Quercetin 3-O-glucosyl-rhamnosyl-glucoside | e-Viniferin |
| Quercetin 3-O-glucosyl-xyloside | Pallidol |
| Quercetin 3-O-glucuronide | Piceatannol |
| Quercetin 3-O-rhamnoside | Piceatannol 3-O-glucoside |
| Quercetin 3-O-rhamnosyl-galactoside | Pinosylvin |
| Quercetin 3-O-rhamnosyl-rhamnosyl- | Pterostilbene |
| glucoside | |
| Quercetin 3-O-rutinoside | Resveratrol |
| Quercetin 3-O-sophoroside | Resveratrol 3-O-glucoside |
| Quercetin 3-O-xyloside | Resveratrol 5-O-glucoside |
| Quercetin 3-O-xylosyl-glucuronide | |

### Resveratrol

Resveratrol is a polyphenolic compound typically found concentrated in the seeds and skins of grapes and berries. It has a strong anti-inflammatory and antioxidant effect. It is indicated to: lower blood pressure and promote heart health, balance blood lipids—decrease LDL (bad cholesterol) & increase HDL (good cholesterol), promote longevity by activating genes associated with warding off the diseases of aging, protect the aging brain by slowing age-related cognitive decline, increase insulin sensitivity and prevent complications from diabetes, relieve symptoms of osteoarthritis by reducing inflammation in joints and providing pain relief, and can even aide the impacts of cancer in that it has been shown to kill colon, skin, breast, and prostate cancer cells in animal cell studies.

The metabolic pathways resveratrol impacts include mTOR, SIRT1/PGC1alpha, autophagy, and proteostasis.

In some embodiments, the present disclosure provides compositions comprising, consisting of, or consisting essentially of, a tri-molecular complex of resveratrol, a polysaccharide, and a protein. The tri-molecular complex can be produced using a method disclosed herein. In various embodiments, the polysaccharide is covalently bound to the protein via a covalent bond between a carboxyl group on the polysaccharide and an amine group on the protein. In some embodiments, the polysaccharide is a derivative of a polysaccharide and in some embodiments, the protein is a hemp protein.

### Berberine

Berberine is an alkaloid phytochemical primarily found in plants of the genus Berberis. It is indicated to: reduce blood sugar thus being helpful for diabetes, decrease insulin resistance, increases glycolysis, decrease gluconeogenesis in the liver, slow the breakdown of carbohydrates in the gut, increase the number of beneficial bacteria in the gut, assist with weight loss by helping a subject lose weight, lower blood lipids and thus the risk of heart disease, and can even aide the impacts of cancer through its impact on the c-myc metabolic pathway, which is indicated in 70% of human cancers including ovarian, breast, colorectal, pancreatic, gastric, and uterine cancers.

The metabolic pathways berberine impacts include AMPK signaling pathway and c-myc.

In some embodiments, the present disclosure provides compositions comprising, consisting of, or consisting essentially of, a tri-molecular complex of berberine, a polysaccharide, and a protein. The tri-molecular complex can be produced using a method disclosed herein. In various embodiments, the polysaccharide is covalently bound to the protein via a covalent bond between a carboxyl group on the polysaccharide and an amine group on the protein. In some embodiments, the polysaccharide is a derivative of a polysaccharide and in some embodiments, the protein is a hemp protein.

In one embodiment, the present disclosure provides a composition comprising a tri-molecular complex of berberine, alginate and pea protein, wherein the alginate and pea protein are covalently bound to each other and then non-covalently complexed with berberine.

### Curcumin

Curcumin is a phytochemical found in turmeric (Curcumin longa), a type of ginger, and is one of three curcuminoids present in turmeric. Curcumin typically exists in two tautomeric forms, a keto and an enol form, and will regularly tautomerize back and forth between such forms. It is a well-known anti-inflammatory agent and is well known to demonstrate anti-neoplastic effects. It is indicated: as a powerful anti-inflammatory agent, as a protectant against heart disease by improving endothelial function, to assist with the effects of cancer and has been shown to help prevent colorectal, pancreatic, prostate, and breast cancers and to help ease gastric disorders, to ease symptoms of osteoarthritis by lowering pain, as an antioxidant where it protects a subject's body from free radicals, to improve brain health by increasing Brain Derived Neurotrophic Factor (BDNF), and as a preventative agent of Alzheimer's Disease.

The metabolic pathways it impacts include mTOR, NFκb, NRF2 and autophagy/proteostasis.

In some embodiments, the present disclosure provides compositions comprising, consisting of, or consisting essentially of, a tri-molecular complex of a curcuminoid (e.g., curcumin), a polysaccharide, and a protein. The tri-molecular complex can be produced using a method disclosed herein. In various embodiments, the polysaccharide is covalently bound to the protein by a covalent bond between a carboxyl group on the polysaccharide and an amine group on the protein. In some embodiments, the polysaccharide is a derivative of a polysaccharide and in some embodiments, the protein is a hemp protein.

In some embodiments, the present disclosure provides a composition comprising a tri-molecular complex of curcumin, alginate and hemp protein, wherein the alginate and hemp protein are covalently bound to each other and then non-covalently complexed with curcumin. In some embodiments, the curcumin of the tri-molecular complex is non-covalently bound to, or otherwise associated with, one or more of SEQ ID NO:1-SEQ ID NO:14 within the hemp protein. In other embodiments, the present disclosure provides a composition comprising a tri-molecular complex of curcumin, alginate and one or more of the peptides of SEQ ID NO:1-SEQ ID NO: 14, wherein the alginate and the one or more peptides are covalently bound to each other and then non-covalently complexed with curcumin. In some embodiments, the hemp protein is highly globular, such as a globular hemp protein. In some embodiments, the alginate is replaced with fructooligosacharride, an indigestible fiber.

### Quercetin

Quercetin is a flavanol polyphenol found in many fruits and vegetables. It has powerful antioxidant properties. It is indicated: to ease allergy symptoms, as an anti-inflammatory agent, to help ease the effects of cancer by slowing the growth of cancer cells in the prostate, liver, lung, breast, Bladder, colon, and/or ovary, to improve brain health by helping to slow the onset of Alzheimer's Disease and dementia, and to help lower blood pressure.

The metabolic pathways it impacts include mTOR, AMPK, and Senolytics.

In some embodiments, the present disclosure provides compositions comprising, consisting of, or consisting essentially of, a tri-molecular complex of quercetin, a polysaccharide, and a protein. The tri-molecular complex can be produced using a method disclosed herein. In some embodiments, the polysaccharide is covalently bound to the protein via a covalent bond between a carboxyl group on the polysaccharide and an amine group on the protein. In some embodiments, the polysaccharide is a derivative of a polysaccharide, and, in some embodiments, the protein is a hemp protein.

In some embodiments, the present disclosure provides a composition comprising a tri-molecular complex of quercetin, alginate and pea protein, wherein the alginate and pea protein are covalently bound to each other and then non-covalently complexed with quercetin.

### Epigallocatechin Gallate (EGCG)

Epigallocatechin gallate, commonly known as EGCG, is a catechin polyphenol found in berries, tea, and cocoa. It is indicated: as an antioxidant, as an anti-inflammatory, to help improve heart health by reducing blood pressure and cholesterol, to assist with weight loss, and to help improve brain health.

The metabolic pathways it impacts include AMPK, NFκb, Autophagy, and Senolytics—HSP90 inhibition.

In some embodiments, the present disclosure provides compositions comprising, consisting of, or consisting essentially of, a tri-molecular complex of epigallocatechin gallate (EGCG), a polysaccharide, and a protein. The tri-molecular complex can be produced using a method disclosed herein. In some embodiments, the polysaccharide is covalently bound to the protein via a covalent bond between a carboxyl group on the polysaccharide and an amine group on the protein. In some embodiments, the polysaccharide is a derivative of a polysaccharide and in some embodiments, the protein is a rice protein.

### Silymarin

Silymarin is a flavonolignan extracted from the milk thistle Silybum marianum (L.). It is indicated: as a hepatoprotective agent by promoting liver health, to improve brain health by helping to prevent age-related decline in brain function, to improve bone health by helping to prevent osteoporosis, as an aid to treatment of certain cancers, to help improve skin health by helping to prevent acne and associated skin lesions or scarring, and to assist with diabetes by improving insulin sensitivity and decreasing blood sugar.

The metabolic pathways it impacts include mTOR, AMPK, NFκb, and FOXO3.

In some embodiments, the present disclosure provides compositions comprising, consisting of, or consisting essentially of, a tri-molecular complex of silymarin, a polysaccharide, and a protein. The tri-molecular complex can be produced using a method disclosed herein. In some embodiments, the polysaccharide is covalently bound to the protein via a covalent bond between a carboxyl group on the polysaccharide and an amine group on the protein. In some embodiments, the polysaccharide is a derivative of a polysaccharide and in some embodiments, the protein is a hemp protein.

### Ursolic Acid

Ursolic acid is a lipophilic pentacyclic triterpenoid that is abundant in apple peel. It is indicated: to help build muscle mass and inhibit muscle damage, to help reduce body weight, BMI and waist circumference, to improve fasting glucose and insulin sensitivity, to aid in the browning of fat by helping regulate white and brown adipose tissue, and can even aid with certain cancers by inhibiting a number of cancer cell types.

The metabolic pathways it impacts include mTOR (P13K/AKT), NFκb, AMPK, Polyol, and UCP Induction.

In some embodiments, the present disclosure provides compositions comprising, consisting of, or consisting essentially of, a tri-molecular complex of ursolic acid, a polysaccharide, and a protein. The tri-molecular complex can be produced using a method disclosed herein. In some embodiments, the polysaccharide is covalently bound to the protein via a covalent bond between a carboxyl group on the polysaccharide and an amine group on the protein. In some embodiments, the polysaccharide is a derivative of a polysaccharide and in some embodiments, the protein is a hemp protein.

### Alginates

An alginate is a gelatinous polysaccharide extract from brown algae and a salt of alginic acid, a linear polymer of mannuronic and glucuronic acids, found in the cell walls of algae.

The alginate in a composition and/or complex provided by the present disclosure can vary. In various aspects, the polyphenol provided in the disclosed compositions is selected from all curcuminoids, quercetin, resveratrol, 6-shogoal, fisetin, naringin, apogenin, pterostilbene, baicalin, berberine, xanthohumol, boswellia, all withanolides, all green tea catechins including EGCG, and combinations thereof. In some embodiments, the alginate is replaced with fructooligosacharride, an indigestible fiber.

In some embodiments, the alginate may be any alginate, provided that the presence of the alginate aids in increasing the bioavailability of a polyphenol in the complex or composition. In some embodiments, the alginate may be a non-digestible alginate. In other embodiments, the alginate is not digested in the upper gastrointestinal (GI) tract. In some embodiments, the alginate may comprise one or more carboxyl groups (e.g., is a carboxylate anion). In some embodiments, the alginate is capable of undergoing a Maillard reaction, which may result from an increase in the pH of the alginate's environment. In some embodiments, the alginate is esterified to a molecule that may be an alcohol. One example of such an alcohol is propylene glycol. In some embodiments, the alginate is at least 50% esterified, at least 60% esterified, at least 70% esterified, at least 80% esterified, at least 90% esterified, or less than 100% esterified. In some embodiments, the alginate is an alginate derivative. In various embodiments, the alginate is propylene glycol alginate. In one embodiment, the alginate is propylene glycol alginate having a molecular weight less than 10 kDa, for example 6 kDa, 5 kDa, 4 kDa, 3 kDa, 2 kDa, 1 kDa or less than 1 kDa. In some embodiments, the alginate is low viscosity as measured by the G to M ratio and is between 50% and 90% esterified.

### Proteins

The protein in a composition and/or complex provided by the present disclosure can vary. In various aspects, the protein in the disclosed compositions is a vegetable protein selected from hemp, soy, coconut, pumpkin, watermelon, sunflower, pea, lentils, white and brown rice (germ and non-germ), flax, oat, wheat, fishmeal, albumin, casein, whey, collagen from all sources, and combinations thereof.

In some embodiments, the protein may be either a protein, a peptide, or a plurality of peptides, provided that the presence of the protein, peptide or plurality of peptides aids in increasing the bioavailability of a polyphenol in the complex or composition. In some embodiments, the protein is a globular protein or a peptide thereof. As used herein, a “peptide” refers to short chain amino acid molecules of between 5 and 100 amino acids linked by peptide bonds. In some embodiments, a protein or peptide comprises one or more hydrophobic stretches of amino acids. A hydrophobic stretch of amino acids may comprise, or consist of, at last 7 hydrophobic amino acids, at least 10 hydrophobic amino acids, at least 20 hydrophobic amino acids, or at least 25 hydrophobic amino acids, which may or may not be contiguous amino acids. In some embodiments, the protein is derived from a globular protein. In some embodiments, the peptide is derived from a globular protein. In various embodiments, the peptide is about 10-100 amino acids in length, about 10-50 amino acids in length, or about 10-25 amino acids in length. In some embodiments, the protein is a plant protein. In some embodiments, the protein is a vegetable protein. In some embodiments, the protein is selected from the group consisting of a hemp protein, a soy protein, a coconut protein, a pumpkin protein, a watermelon protein, a sunflower protein, a pea protein, a lentil protein, a brown rice protein, a flax protein, an oat protein, a wheat protein, a whey protein, a fishmeal protein, collagen, albumin, and casein. In some embodiments, the protein comprises, or consists of, an amino acid sequence selected from the group consisting of SEQ ID NO:1-SEQ ID NO:14. The amino acid sequences of SEQ ID NO:1-SEQ ID NO:14 occur in one or more hemp proteins.

### Dosage Forms

In certain aspects, compositions and/or tri-molecular complexes of the disclosure may comprise additional agents to provide a form suitable for administration to an individual subject. Such agents may be described as biologically inactive and can be administered to subjects without causing deleterious interactions with the active agents. Examples of suitable agents include, but are not limited to, excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Additional examples of suitable agents include, but are not limited to, vitamins, minerals, trace elements, oils (e.g., olive oil) antioxidants, pharmaceutical agents (i.e., drugs), fiber, buffering agents, antacids, stabilizers, sweetening agents, flavoring agents, dyes, or coloring agents. Examples of such additional agents are disclosed in US Patent Publication No. 2013/0095204, US Patent Publication No. 2007/0077279 and U.S. Pat. No. 9,839,624.

Compositions of the disclosure can take the form of solutions, suspensions, emulsions, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see, e.g., Grosswald et al., U.S. Pat. No. 5,698,155). Preferred compositions provided by the present disclosure are formulated for oral delivery, which can be oral sustained administration.

Compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, where in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds and compositions of the present disclosure. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcarnitines and the like may be added.

Compositions may also take a form for administration via other routes. For buccal administration, the compositions may take the form of tablets, lozenges, etc. formulated in conventional manner. Liquid drug formulations suitable for use with nebulizers and liquid spray devices and EHD aerosol devices will typically include a composition provided by the present disclosure with a pharmaceutically acceptable vehicle. Preferably, the pharmaceutically acceptable vehicle is a liquid such as alcohol, water, polyethylene glycol or a perfluorocarbon. Optionally, another material may be added to alter the aerosol properties of the solution or suspension of compositions of the present disclosure. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611). A composition provided by the present disclosure may also be formulated in rectal or vaginal compositions such as suppositories or retention enemas, e.g., containing conventional suppository bases such as cocoa butter or other glycerides. In addition to the formulations described previously, a composition provided by the present disclosure may also be formulated as a depot preparation. Such long acting formulations may be administered by implantation (for example subcutaneously or intramuscularly) or by intramuscular injection. Thus, for example, a composition provided by the present disclosure may be formulated with suitable polymeric or hydrophobic materials (for example as an emulsion in an acceptable oil) or ion exchange resins, or as sparingly soluble derivatives, for example, as a sparingly soluble salt.

### Methods of Making Compositions

As noted herein, the present disclosure provides compositions comprising a polyphenol, a polysaccharide and either a protein or one or more peptides provided by SEQ ID NO: 1-SEQ ID NO: 14, in which the polyphenol, the polysaccharide, and the protein/one or more peptides are combined to form a tri-molecular complex. Polyphenols like curcumin are hydrophobic and thus offer low absorption through digestion when taken in an unaltered, raw form. It is also well known that many proteins have a hydrophobic “interior” portion and an hydrophilic “exterior” portion. The compositions of the present disclosure take advantage of these differences in hydrophilicity in order to enhance the bioavailability of a polyphenol.

In one aspect, a tri-molecular complex or composition provided by the present disclosure, comprising a polyphenol and one or more proteins covalently bound to a polysaccharide, can be prepared as follows: In an ethanol (or similar organic) environment, the polyphenol (e.g., curcumin) is soluble. A sample of the polysaccharide-protein is unfolded (denatured) in that ethanolic/organic environment, allowing the hydrophobic portions of each denatured protein to begin to associate with each other. Once that happens, these hydrophobic associated portions tend to fall out of solution. The hydrophilic portions will then associate with, or “encapsulate” the polyphenol molecule—making the hydrophobic portion of this molecule more hydrophilic. Once this association/encapsulation process is complete and the polyphenol-protein-polysaccharide complex is prepared, the complex will demonstrate enhanced hydrophilicity, resulting in increased absorption in the digestion process, thereby improving bioavailability.

In various aspects, compositions provided by the present disclosure can be prepared via the coating of a polysaccharide with protein via transacylation reactions. Such reactions involve nucleophilic addition—elimination between an esterified carboxylic group and an amino group, yielding an amide and an alcohol. The reaction takes place when amino groups become uncharged under alkaline conditions (—NH₂).

Esterified carboxylic acids present on the polysaccharide bind any amino group on the protein, with a preference for lysine or the amino terminus of the protein. At high pH (about pH 11), amine groups lose a hydrogen, enabling those amines to react with the esterified carboxylic acid and make a covalent bond. This combination of a covalently attached, hydrophilic polysaccharide and a hydrophobic core of a protein binds well to a polyphenol. This esterification reaction catalyzes this reaction very quickly (20 fold faster) and eliminates the need for heat, which would denature the product, resulting in a higher yield of viable composition.

In several aspects, compositions provided by the present disclosure can be prepared by first combining a polysaccharide and a protein/one or more peptides to form a covalent, bi-molecular complex wherein the polysaccharide is covalently bound to the protein/one or more peptides. Formation of the covalent bond can occur via a trans-acylation reaction whereby a covalent bond is formed between a carboxyl group on the polysaccharide and an amine group on the protein/one or more peptides. In some embodiments, formation of a covalent bond between the polysaccharide and the protein/one or more peptides comprises forming a mixture comprising the polysaccharide and the protein/one or more peptides, increasing the pH of the mixture for a period of time, and decreasing the pH of the mixture, thereby forming a bi-molecular complex in which the polysaccharide is covalently bound to the protein/one or more peptides via transacylation. In such a method, the pH may be increased to at least about pH 9, at least about pH 10, at least about pH 11, or at least about pH 12 and, after the period of time, decreased to a neutral pH of between about 6.8 to about 7.2. In such a method, the mixture may lack available calcium. Once the bimolecular complex is formed between the polysaccharide and the protein/one or more peptides, the mixture may be dehydrated to form a power, commonly referred to as a carrier or carrier powder.

Once a bi-molecular complex has been formed by the polysaccharide and the protein/one or more peptides, the polyphenol may be contacted with the bi-molecular complex, where it will non-covalently associate with the hydrophobic regions of the protein/one or more peptides, forming a tri-molecular complex of the polyphenol, the polysaccharide, and the protein/one or more peptides. Without being bound by theory, it is believed that, at least some polyphenols, bind to one or more hydrophobic pockets in the interior of the globular protein of the bi-molecular complex. Thus, in some embodiments, formation of a tri-molecular complex of the polyphenol, the polysaccharide, and the protein/one or more peptides, may comprise denaturing conditions. In some embodiments, prior to contacting the polyphenol with the bi-molecular complex, the polyphenol may be introduced into an organic solvent, which may be contacted with the bi-molecular complex. Alternatively, in other embodiments, prior to contacting the polyphenol with the bi-molecular complex, the bi-molecular complex (or carrier), may be subject to denaturing conditions selected from heat and/or organic solvents, such as an organic alcohol (e.g., methanol, ethanol, etc.). Contact of the polyphenol with the bi-molecular complex results in formation of the polyphenol/polysaccharide/protein complex. Another example of a method of making the compositions provided by the present disclosure is provided in Example 1.

Methods of the disclosure result in the formation of a tri-molecular complex of a polyphenol, a polysaccharide, and a protein. Incorporation of the polyphenol into such a tri-molecular complex increase the bioavailability of the polyphenol.

### Methods, Dosing

Tri-molecular complexes and compositions provided by the present disclosure may generally be used to prevent or treat various conditions and/or to generally improve the health of a subject. As used herein, “subject” refers to any human or non-human animal. Examples include, but are not limited to, humans, non-human primates, such as chimpanzees, apes and other monkey species; domestic mammals (e.g., dogs and cats); laboratory animals (e.g., mice, rats guinea pigs); birds, and bats. Subjects of any age or race are covered by the present disclosure.

Tri-molecular complexes and compositions provided by the present disclosure are useful for the treatment of any condition or disease that capable of being treated with a polyphenol. Examples of conditions and diseases that may be prevented or treated using the disclosed compositions or tri-molecular complexes include, but are not limited to, pain, high blood pressure, heart disease, high cholesterol (i.e., low density lipids (LDL)), low high density lipids (HDI), lipidemia, diseases of aging, cognitive decline, high blood sugar, insulin resistance, metabolic syndrome, diabetes, complications resulting from diabetes, inflammation, osteoarthritis, cancer, overweight, obesity, high body mass index (BMI), hepatitis, non-alcoholic steatohepatitis (NASH), non-alcoholic fatty liver disease (NAFLD), poor digestion, poor cognition, impaired memory, impaired executive function, Alzheimer's disease, Parkinson's disease, multiple sclerosis, allergy, skin lesions, scaring, acne, benign prostatic hyperplasia, and erectile dysfunction.

The polyphenol is the active agent of a tri-molecular complex and/or composition provided by the present disclosure. As such, the amount (weight amount, concentration, etc.) of a composition or tri-molecular complex suitable for use in a method of treatment, will be determined by the amount of polyphenol for which it is desired to deliver to a subject. Moreover, the desired amount of polyphenol may depend on the nature of the conditions or disease to be treated, and can be determined by standard clinical techniques known in the art. In addition, in vitro or in vivo assays may optionally be employed to help identify optimal dose ranges. In some embodiments, the amount of a composition, or tri-molecular complex, administered will be dependent on, among other factors, the subject being treated, the weight of the subject, the severity of the disease, the manner of administration and the judgment of any prescribing health care provider. In some embodiments, an amount of a composition, or tri-molecular complex, is administered to a subject to deliver an amount of polyphenol in the range of from about 1 microgram (μg) to about 2,000 milligrams (mg). In some embodiments, an amount of a composition, or tri-molecular complex, is administered to a subject to deliver at least 5 μg, at least 10 μg, at least 25 μg, at least 50 μg, at least 100 μg, at least 500 μg, at least 1 mg, at least 5 mg, at least 10 mg, at least 25 mg, at least 50 mg, at least 100 mg, at least 250 mg, at least 50 mg, at least 1000 mg, or at least 2000 mg, to a subject.

In some embodiments, a dose of a composition or tri-molecular complex may be delivered in a pharmaceutical composition by a single administration or by multiple administrations, optionally with controlled release. In some embodiments, a dose of a composition or tri-molecular complex may delivered by oral release administration. In some embodiments, a dose of a composition or tri-molecular complex may be administered once per day, twice per day, or on any schedule necessary to achieve the desired effect.

In some embodiments, a method of modulating a biological system in a subject is provided, the method comprising administering a composition or a tri-molecular complex of the disclosure to the subject. In these embodiments, modulating a biological system comprises an effect selected from reducing inflammation, lowering blood pressure, balancing blood lipids, decreasing the concentration of low density lipids (LDL), increasing the concentration of high density lipids (HDL), activating genes associated with diseases of aging, improving memory, increasing cognition, increasing executive function, decreasing insulin resistance, increasing insulin sensitivity, preventing complications from diabetes, reducing pain, reducing tumor growth, increasing glycolysis, decreasing gluconeogenesis, slowing carbohydrate catabolismaltering a microbiome, reducing body weight, reducing body mass index (BMI), increasing weight, reducing free radicals, altering oxidation-reduction status, increasing the production of brain derived neurotrophic factor (BDNF), reducing allergy symptoms, reducing or preventing acne, reducing scarring, reducing skin lesions, increasing lipid metabolism, improving sexual function, improving libido, and combinations thereof.

In some embodiments, a method of protecting a subject against a condition or a disease is provided, comprising administering to the individual a tri-molecular complex or a composition provided by the disclosure. The subject may, but need not, be at risk for developing the condition or disease. The condition or disease is selected from the group consisting of pain, high blood pressure, heart disease, high cholesterol (i.e., low density lipids (LDL)), low high density lipids (HDL), lipidemia, diseases of aging, cognitive decline, high blood sugar, insulin resistance, metabolic syndrome, diabetes, complications resulting from diabetes, inflammation, osteoarthritis, cancer, overweight, obesity, high body mass index (BMI), hepatitis, non-alcoholic steatohepatitis (NASH), non-alcoholic fatty liver disease (NAFLD), poor digestion, poor cognition, impaired memory, impaired executive function, Alzheimer's disease, Parkinson's disease, multiple sclerosis, allergy, skin lesions, scaring, acne, benign prostatic hyperplasia, erectile dysfunction, and combinations thereof. In some embodiments, the tri-molecular complex or composition may be administered in conjunction with one or more other treatments for the condition or disease.

In some embodiments, a method of treating a condition or a disease in a subject comprises administering to the subject a tri-molecular complex or a composition provided by the disclosure. The condition or disease is selected from the group consisting of pain, high blood pressure, heart disease, high cholesterol (i.e., low density lipids (LDL)), low high density lipids (HDL), lipidemia, diseases of aging, cognitive decline, high blood sugar, insulin resistance, metabolic syndrome, diabetes, complications resulting from diabetes, inflammation, osteoarthritis, cancer, overweight, obesity, high body mass index (BMI), hepatitis, non-alcoholic steatohepatitis (NASH), non-alcoholic fatty liver disease (NAFLD), poor digestion, poor cognition, impaired memory, impaired executive function, Alzheimer's disease, Parkinson's disease, multiple sclerosis, allergy, skin lesions, scaring, acne, benign prostatic hyperplasia, erectile dysfunction, and combinations thereof. In some embodiments, the tri-molecular complex or composition may be administered in conjunction with one or more other treatments for the condition or disease.

### EXAMPLES

The following examples provide those of ordinary skill in the art with a description of how to make and use the subject matter disclosed herein and are not intended to limit the scope of what the inventors regard as inventive nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1. Production of a Curcumin-Containing Tri-Molecular Complex

### Preparation of an Alginate-Hemp Protein Bimolecular Complex

Materials: 800 mL reverse-osmosis purified (RO) water
   30 g propylene glycol alginate (Biofoam K, BSG Craft Brewing, Shakopee MN)
   250 g hemp protein (Nutiva, Richmond, CA)
   100 mL 1.0M NaOH (LabChem, Zelienople, PA)
   100 mL HCl (LabChem, Zelienople, PA)

Procedure: heat 800 mL of RO water to 70° C. (150° F.) in the mixing bowl of a KitchenAid-style mixer. Evenly distribute 30 g of propylene glycol alginate in the heated RO water and let stand, at temperature, for 3 minutes. Mix the RO water and propylene glycol alginate together, using low speed, for about 3-5 min until a uniform mixture is achieved.

Add 250 g of hemp protein to the uniform mixture of RO water and propylene glycol alginate. Mix together. Add 100 mL 1.0M NaOH and mix for about 3-5 minutes. Let stand for about 10 minutes. Add 100 mL 1.0M HCl and mix for about 3-5 minutes. Let stand for about 7 minutes.

Distribute the resulting mixture evenly into 8″×10″ metal pans and load the pans into an oven or heating cabinet pre-heated to 190° F. Heat for about 5 hours. After 5 hours, reduce the temperature to 170° F. Heat for at least 8 hours, preferably overnight.

The temperature is controlled such that it does not rise above 200° F., as such temperatures can negatively impact the resulting bimolecular complex. Care is taken to ensure that the product is completely dried at the end of the 170 OF heat cycle, with no visible moisture remaining. The resulting product is a dry powder.

### Preparation of a Curcumin-Alginate-Hemp Protein Tri-Molecular Complex

Materials: 1 L ethyl alcohol (EtOH)
   20 g curcumin (95% pure)
   128 g alginate-hemp protein bimolecular complex powder, prepared as above

Procedure: Boil 1 L of EtOH. Add 20 g of curcumin to the boiling EtOH, bring back to a boil, and maintain heat until the mixture is clear. Add 128 g of powdered alginate-hemp protein bimolecular complex (prepared as above) powder to the EtOH-curcumin mixture and stir. Continue stirring, every 5 minutes, for about 1 hour total time.

Remove the resulting mixture to a pan and heat at 170 ºF until the EtOH has completely evaporated and the powder is visually dry. The end product is a curcumin-alginate-hemp protein tri-molecular complex wherein the alginate and hemp protein are covalently bound together and the curcumin is non-covalently complexed with the two.

### Example 2. Bioavailability of Curcumin Formulations

This example illustrates the bioavailability of curcumin after administration of the following formulations to adult human subjects:
1. Hempcur—a bimolecular complex prepared by the instant inventors, comprising curcumin bound to hemp protein.
2. HempCurAlg—an experimental tri-molecular complex produced by the instant inventors, prepared by first binding curcumin to hemp protein, thereafter binding the curcumin-hemp protein complex to alginate, and lyophilizing the curcumin-hemp protein-alginate complex.
3. UltraCür®: a commercially available curcumin supplement, which is curcumin bound to whey protein. UltraCür Curcumin-Whey Complex, derived from 95% pure curcumin solids (Ultra Botanica LLC, Oklahoma City, OK).
4. Algicur: a tri-molecular curcumin-alginate-hemp protein complex provided by the present disclosure, prepared according to the procedure provided in Example 1.
5. NovaSOL®: a commercially available curcumin supplement, which is 7% soluble curcumin (volume per volume (% v/v)) in 93% Tween-80 (% v/v), a synthetic detergent. (NovaSOL®, Millburn, NJ).

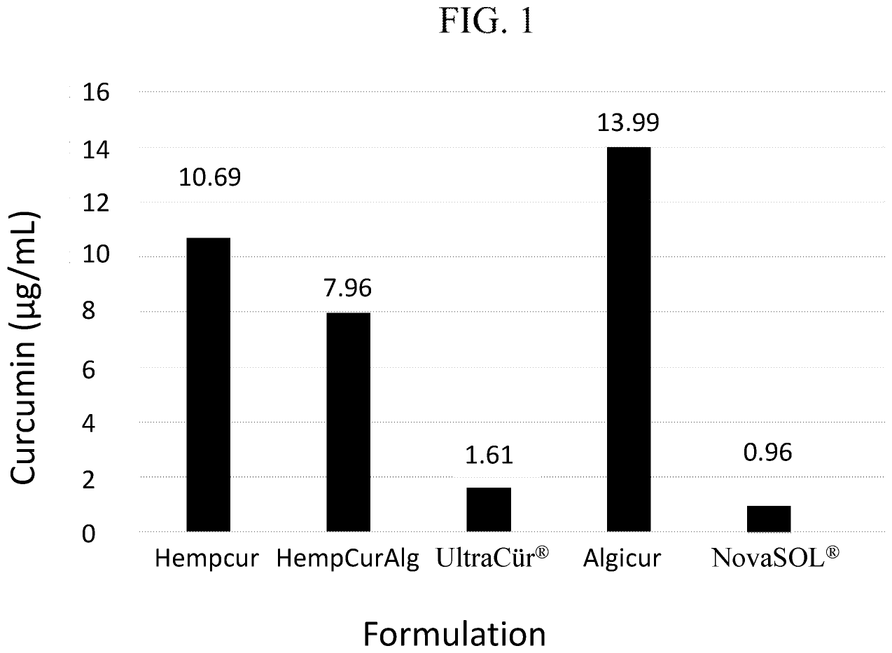

To measure the bioavailability of curcumin in each formulation, each tested formulation was administered to an adult subject, and urine samples were collected, as follows: a sample from the morning void of urine was taken as a control. Following control sample collection, each subject was administered 1.8 grams (g) of one of the formulations shown above; each subject was administered a single formulation only (subject 1 was administered Hempcur, subject 2 was administered HempCurAlg, etc.). After 4 hours, a second urine sample was taken, spun, and the amount of curcumin present in the urine sample determined by fluorescence. Briefly, the sample was subjected to light at a frequency of 430 nM, and emission measured at 530 nM. The resulting emissions are shown in FIG. 1.

FIG. 1 shows that a tri-molecular complex provided by the present disclosure (Algicur), prepared as indicated in Example 1, displayed the highest bioavailability our of all the formulations compared. Three of the four formulations prepared by the instant inventors resulted in elevated bioavailability, however the data clearly show that the Algicur formulation provided significantly improved bioavailability as compared to the two commercially available curcumin products (UltraCür® and NovaSOL®). Further, the bioavailability of curcumin a composition produced according to the disclosed method is significantly higher than the bioavailability of curcumin in UltraCür® and NovaSOL®, both of which are commercially available and are considered to be curcumin formulations showing a high degree of bioavailability of curcumin. The results demonstrate that a formulation of curcumin produced using the disclosed methods yields substantially greater bioavailability of curcumin than the top line products that are commercially available.

This result is surprising because the tri-molecular complex has lower solubility than NovaSOL® (as shown in Example 5 for Curcelite®) but has significantly higher bioavailability. As with solubility, the trimolecular complex surprisingly also decreases inflammation while NovaSOL® increases inflammation, even though Curcelite® has lower solubility.

### Example 3. Clinical Trial Assessing the Bioavailability of Leading Phytonutrient Products

Study Description: This study tests various phytonutrient-based, curcumin-containing supplements to see which gets absorbed into the body the best. The supplements are 120 mg of curcumin from brands Meriva®, CurcElite®, Lonvida®, UltraCur®, NovaSOL®. See Table 3 for further description of the products. The study also tests inflammatory markers in the urine to determine any change.

Conditions: Inflammation

### Study Design

Study Type: Interventional Primary Purpose: Basic Science
Interventional Study Model: Crossover Assignment
Number of Arms: 5
Masking: Single (Participant)
Allocation: Non-Randomized Enrollment: 15 [Anticipated]

TABLE 2
Arms and Interventions

| Arms | Assigned Interventions |
|---|---|
| Meriva ® (120 mg of curcumin) | Curcumin oral supplement |
| Longvida ® (120 mg of curcumin) | Curcumin oral supplement |
| NovaSOL ® (120 mg of curcumin) | Curcumin oral supplement |
| CurcElite ® (120 mg of curcumin) | Curcumin oral supplement |
| UltraCür ® (120 mg of curcumin) | Curcumin oral supplement |

Outcome Measures: Bioavailability Blood Plasma and Inflammation [Time Frame: 0, 1, 2, 4, and 12 hours]

### Eligibility

Minimum Age: 21 Years Maximum Age: 49 Years
Sex: Male
Inclusion Criteria: Age and gender
Exclusion Criteria: Cannot take any phytonutrient or curcumin supplement five days prior to commencement.

Outcomes: Patients receiving Curcelite® have increased curcumin levels in blood, for example at hours 1, 2, 4, and 12, and have decreased inflammatory markers, for example at hours 1, 2, 4, and 12. Patients receiving the other supplements have no or less increase in curcumin levels in blood, for example at hours 1, 2, 4, and 12, and less decrease (relative to subjects receiving Curcelite®), no decrease, or even increase in inflammatory markers, for example at hours 1, 2, 4, and 12.

### Example 4. Clinical Trial Assessing the Bioavailability of Curcelite® and NovaSOL®

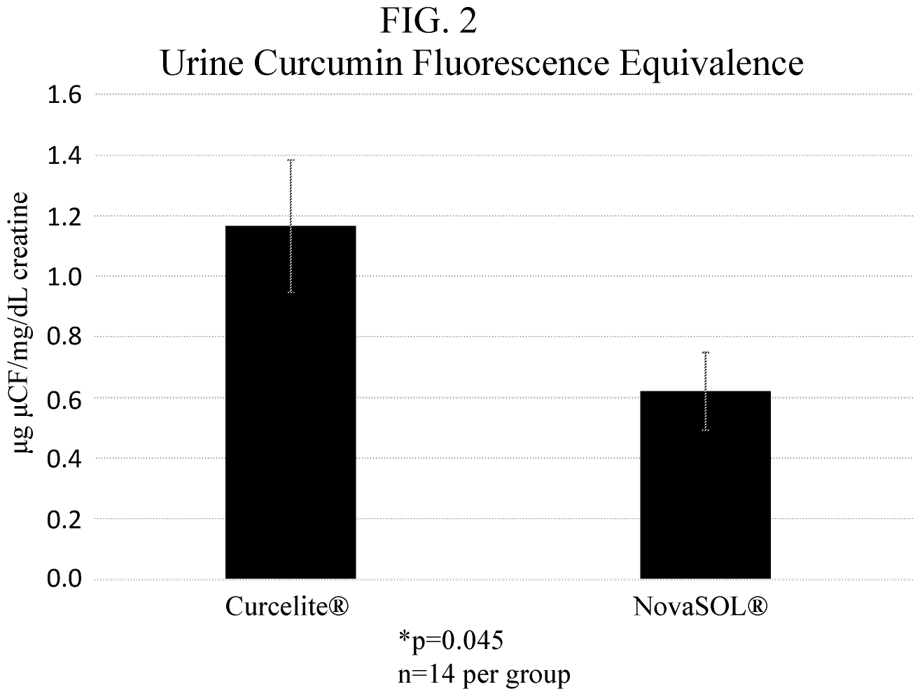

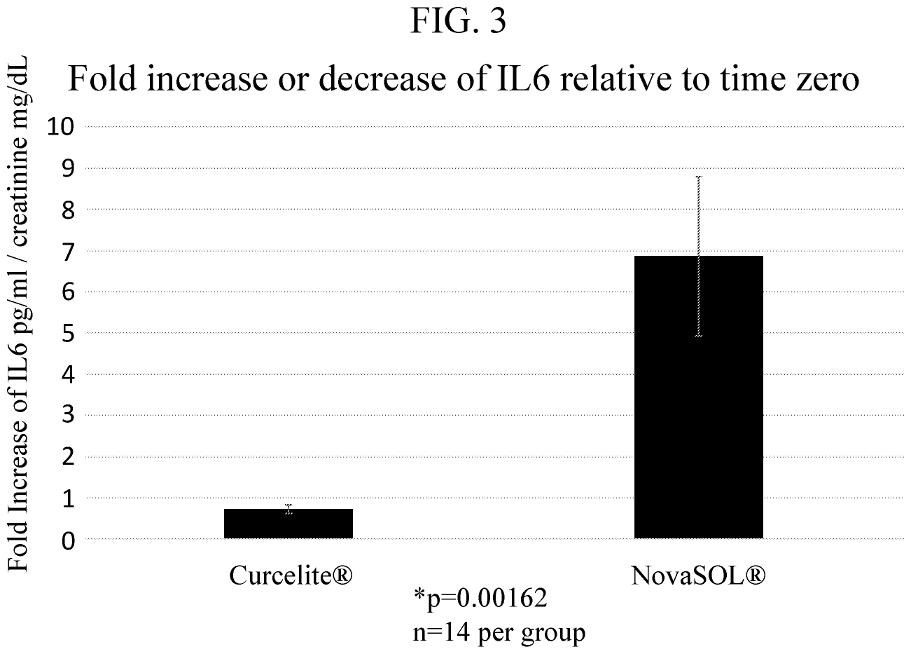

A clinical trial was performed as described in Example 3, except comparing Curcelite® and NovaSOL® at time 0 and time 4 hours (4 hours after administration at time 0). Subjects receiving Curcelite® had at time 4 hours significantly higher levels of curcumin in urine (p=0.045, FIG. 2) and significantly less IL6 (p=0.00162, FIG. 3) in urine than subjects receiving NovaSOL®. Surprisingly, Curcelite® caused a dramatic 28% drop in IL6 level, even though NovaSOL® caused a 686% increase in IL6. This demonstrates the unexpected superiority of Curcelite® in reducing inflammation.

### Example 5. Solubility of Leading Phytonutrient Products

The solubility of several leading phytonutrient products was tested, and the results are shown in Table 3. Briefly, one pill of each was resuspended in water for 2 hours, with vigorous shaking every hour. The resulting samples were centrifuged at 9,000 g for 10 minutes, and the resulting supernatant was transferred to a new tube to remove insoluble material. Curcumin concentration was then determined for each sample. The measured solubility of Curcelite® was 8,727 times higher than that of pure curcumin, even though the Curcelite® formulation hides curcumin fluorescence.

TABLE 3
Solubility of Leading Phytonutrient Products

| | | Solubility |
|---|---|---|
| Product | Product Description | (μg/ml) |
| Curcumin | Pure curcumin | 0.011 |
| Curcuwin ® | cellulosic derivatives complexed with a hydrophobic | 956 |
| | carrier and natural antioxidants (OmniActive Health | |
| | Technologies, Inc., Morristown, NJ, USA) | |
| Curcumin | microcrystalline cellulose combined with soy lecithin | 110 |
| Phytosome | phosphatidylcholine (formerly known as Meriva ® | |
| | Thorne Health Tech, Inc., New York, NY, USA) | |
| Longvida ® | solid lipid curcumin particles composed of | 235 |
| | docosahexaenoic acid (DHA), soy lecithin, stearic acid | |
| | and vitamin C esters (Verdure Sciences ®, Noblesville, | |
| | IN, USA) | |
| BCM-95 ® | curcumin with turmeric oil (Arjuna Natural ® LLC, | 8 |
| | Irving, TX, USA) | |
| CurcElite ® | Trimeric alginate hemp curcumin sandwich (Mara | 96 |
| | Labs, Charlottesville, VA, USA) | |
| NovaSOL ® | 7% curcumin in 93% Tween-80 (Molecular Health | 2356 |
| | Technologies LLC, Millburn, NJ, USA) | |

### Example 6. Characteristics of Curcumin Binding Peptides

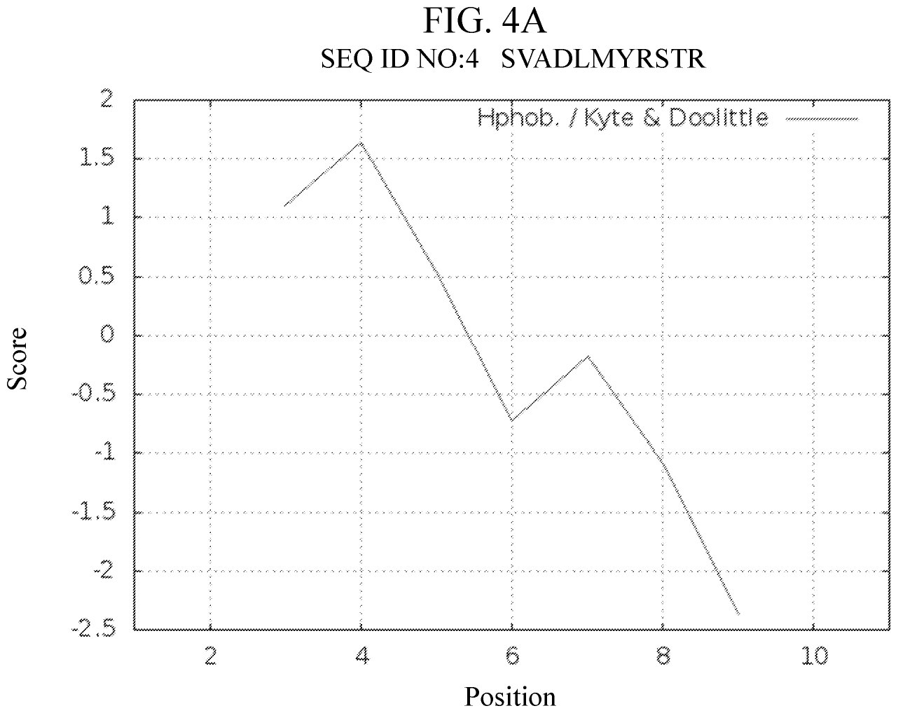

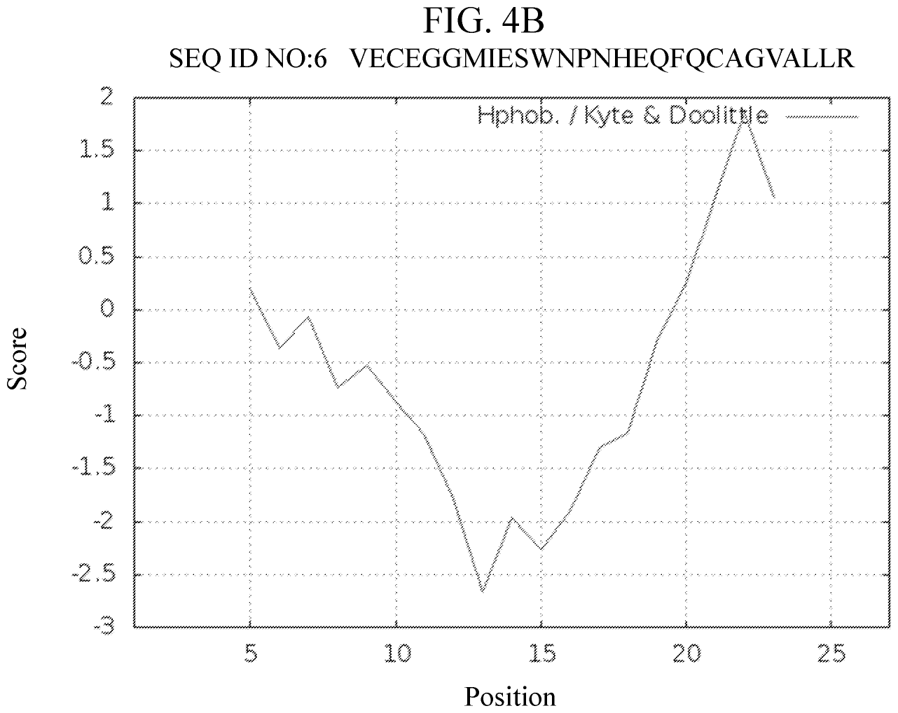

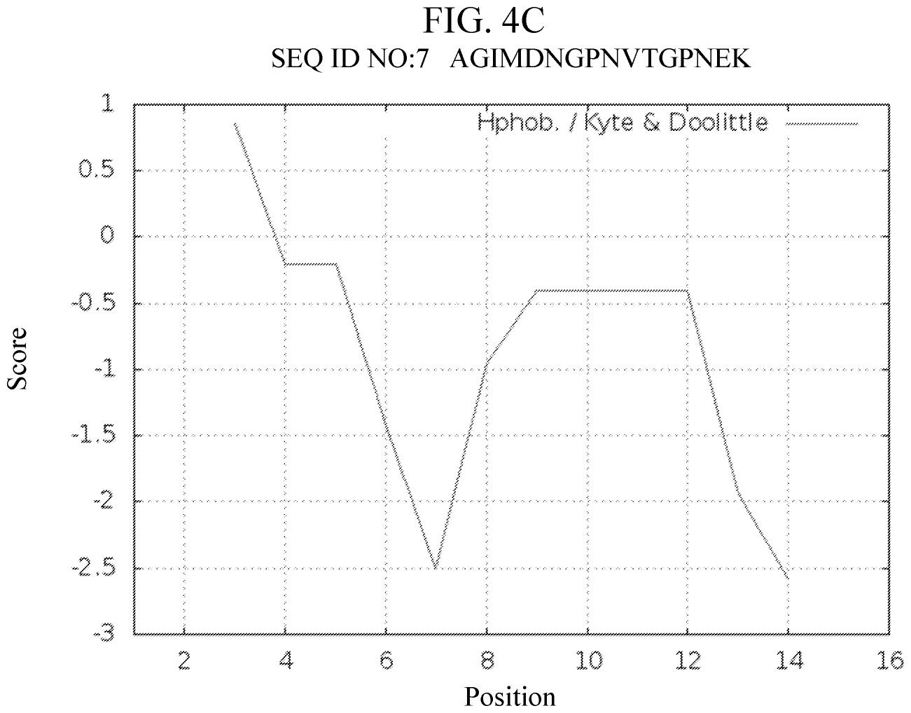

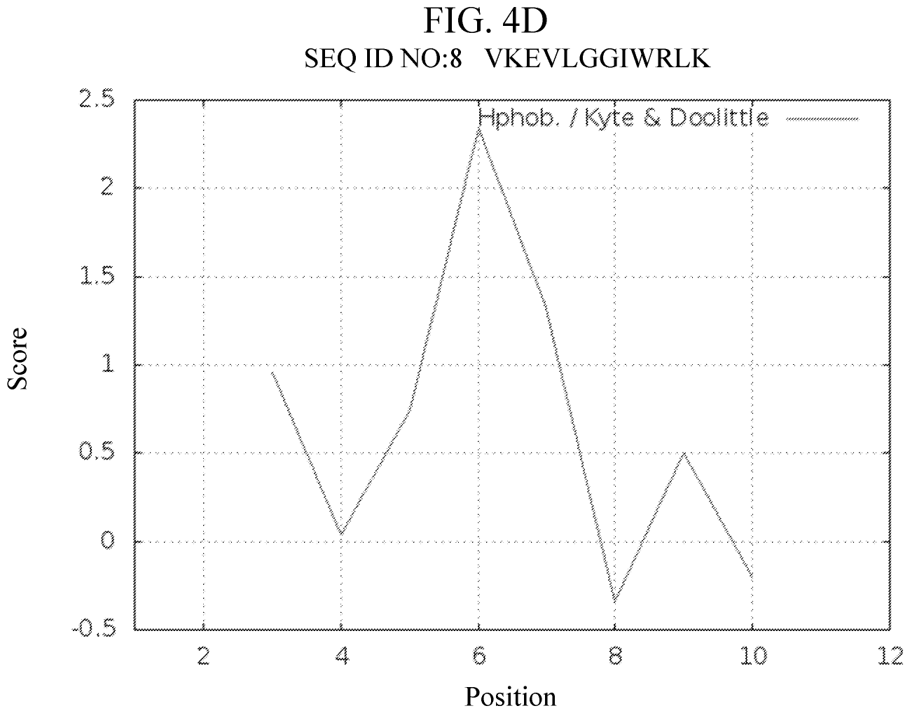

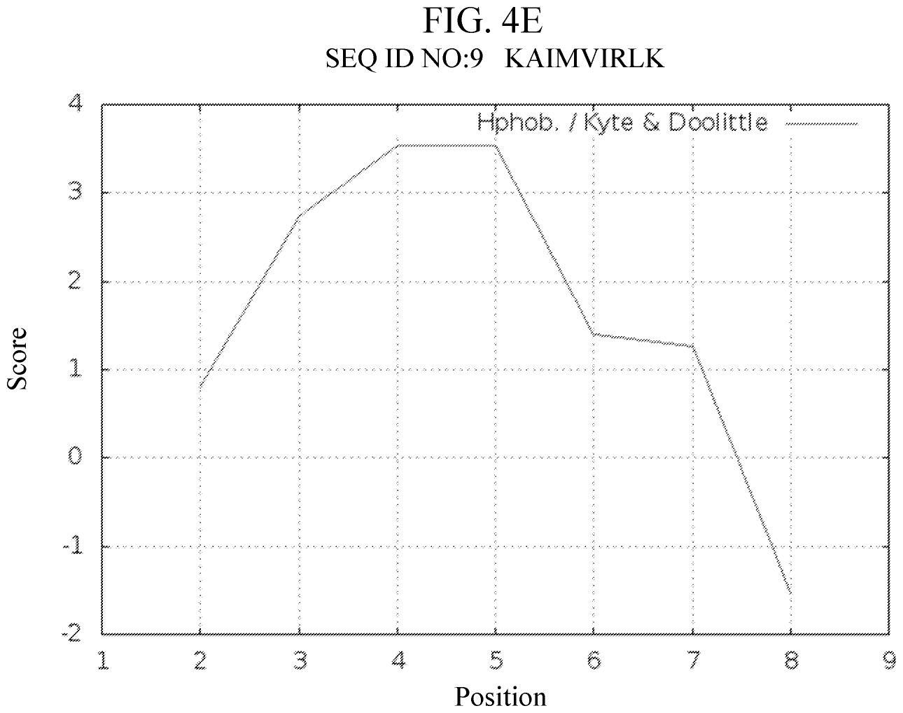

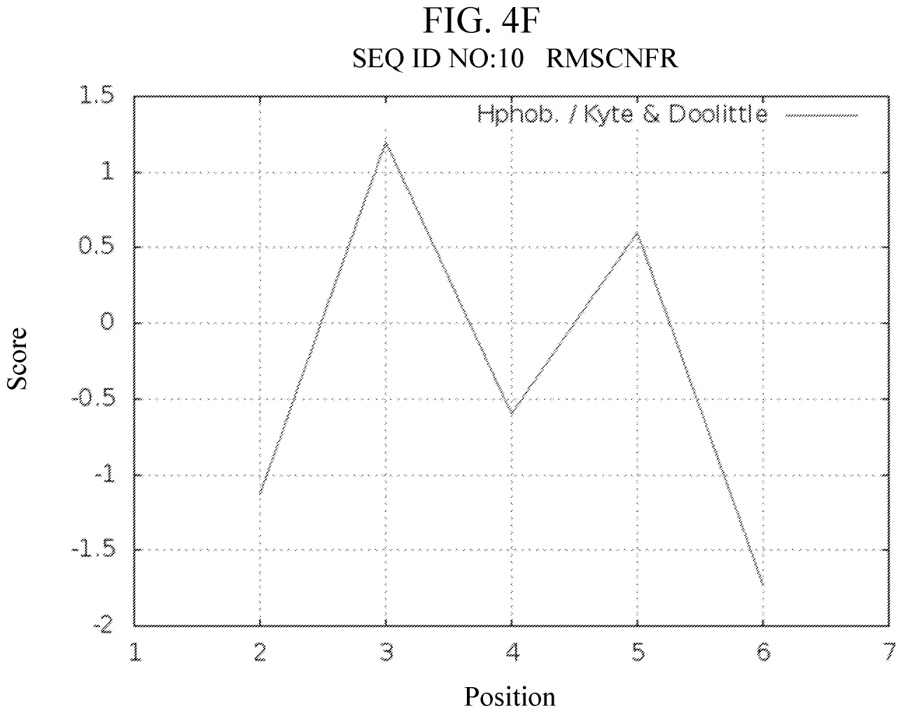

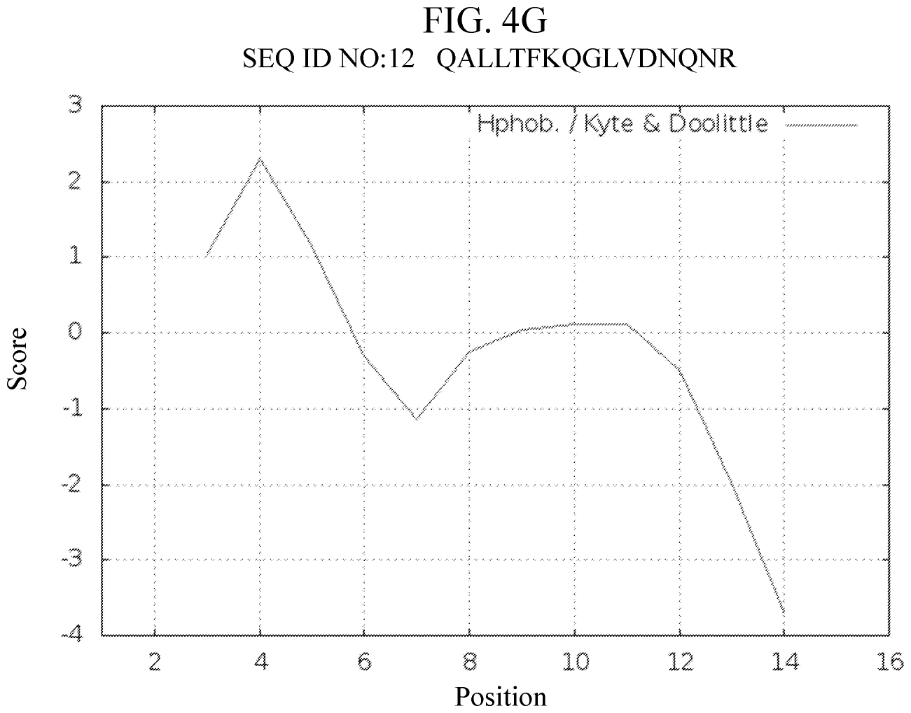

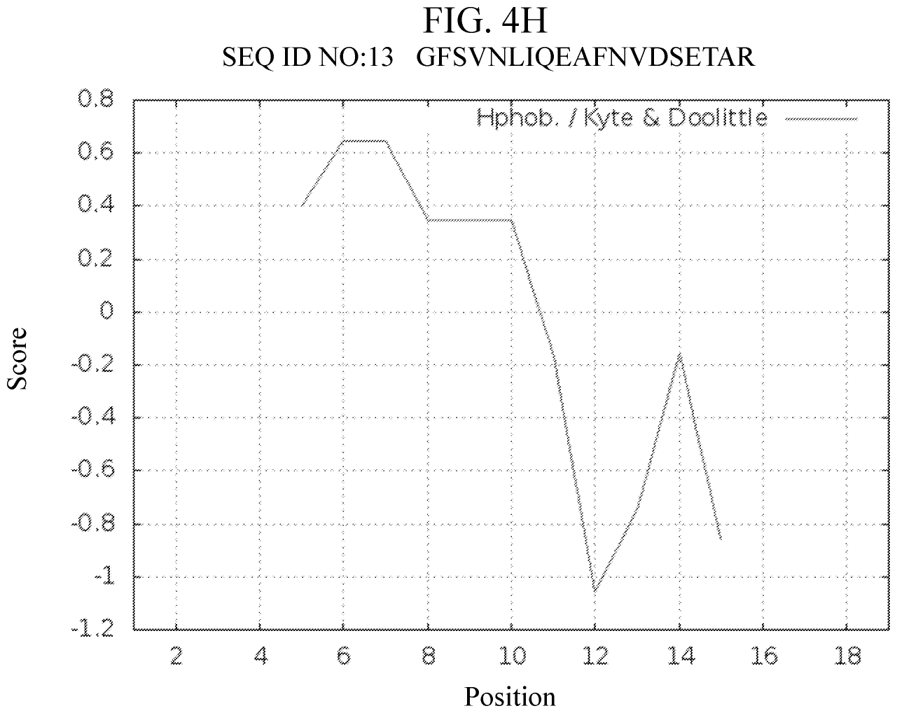

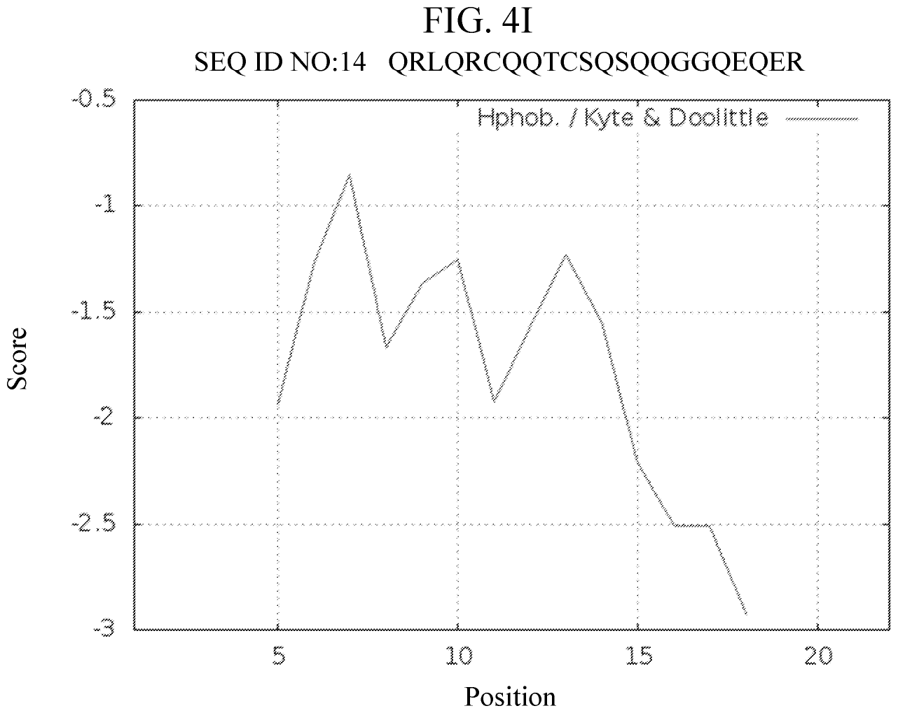

The hydrophobicity of curcumin binding peptides was analyzed. Briefly, curcumin was incubated with a hemp protein lysate containing a mixture of hemp proteins. Curcumin and associated curcumin-binding proteins were isolated from the mixture, digested with trypsin, and analyzed by liquid chromatography (LC) tandem mass spectrometry (MS) (LC-MS/MS). Hydrophobicity profiles were generated for identified peptides with Protscale to determine hydrophobicity (Kyte and Doolittle). Gasteiger E., et al. Protein Identification and Analysis Tools on the ExPASy Server; (In) John M. Walker (ed): The Proteomics Protocols Handbook, Humana Press (2005). pp. 571-607. As shown in FIGS. 4A-4I, each identified peptide contains one or more hydrophobic regions and one or more hydrophilic regions.

We claim:

1. A composition, comprising: a polyphenol, a protein, and a polysaccharide, wherein:
the polysaccharide is bound to the protein by a covalent bond between a carboxyl group on the polysaccharide and an amine group on the protein;
the polyphenol is non-covalently complexed with the covalently bound polysaccharide and protein; and
the bioavailability of the polyphenol in the composition is greater than the bioavailability of the polyphenol in a composition lacking the covalently bound polysaccharide and protein.

2. The composition of claim 1, wherein the polyphenol is selected from the group consisting of a turmeric extract, curcuminoids, curcumin, quercetin, resveratrol, 6-shogaol, fisetin, naringin, apigenin, pterostilbene, baicalin, berberine, silibinin (silybin), Silymarin, ursolic acid, xanthohumol, Boswellia, catechin, epigallocatechin gallate (EGCG), enterodiol, enterolactone, and withanolides.

3. The composition of claim 1, wherein the polyphenol is selected from the group consisting of resveratrol, berberine, curcumin, quercetin, EGCG, silymarin, and ursolic acid.

4. The composition of claim 1, wherein the polyphenol is a polyphenol listed in Table 1, or a derivative thereof.

5. The composition of any one of claims 1-4, wherein the polyphenol is curcumin.

6. The composition of any one of claims 1-5, wherein the polyphenol is berberine.

7. The composition of any one of claims 1-6, wherein the polyphenol is quercetin.

8. The composition of any one of claims 1-4, wherein the polyphenol affects one or more biochemical pathways.

9. The composition of claim 8, wherein the one or more biochemical pathways are selected from the group consisting of the mechanistic target of rapamycin (mTOR) pathway, the Sirtuin 1 (SIRT1) pathway, a peroxisome proliferator-activated receptor-gamma coactivator (PGC)-1alpha pathway, the autophagy/proteostasis pathway, an AMP-activated protein kinase (AMPK) pathway, a c-Myc pathway, a nuclear factor-κB (NF-κB) pathway, a nuclear factor erythroid 2-related factor 2 (Nrf2) pathway, a forkhead box O-3 (FoxO3) pathway, an uncoupling protein 1 (UCP-1) pathway, a signal transduction pathway, a pathway involved in the clearance of senescent cells, and combinations thereof.

10. The composition of any one of claims 1-9, wherein the polysaccharide is selected from the group consisting of alginate, pectin, fructooligosacharride (FOS), galactooligosaccharides (GOS), and starch.

11. The composition of any one of claim 10, wherein the starch is potato starch.

12. The composition of any one of claims 1-11, wherein the protein is selected from the group consisting of a hemp protein, a soy protein, a coconut protein, a pumpkin protein, a watermelon protein, a sunflower protein, a pea protein, a lentil protein, a brown rice protein, a flax protein, an oat protein, a wheat protein, a whey protein, a fishmeal protein, collagen, albumin, and casein.

13. The composition of any one of claims 1-12, wherein the protein is a globular protein, or a peptide thereof.

14. The composition of claim 13, wherein the globular protein, or peptide thereof, comprises one or more hydrophobic stretches of amino acid residues.

15. The composition of claim 13 or claim 14, wherein the protein, or peptide thereof, comprises one or more amino acid sequences selected from the group consisting of SEQ ID NO:1-SEQ ID NO:9.

16. A method of making the composition of any one of claims 1-15, comprising:
coating the polysaccharide with the protein via transacylation reactions by:
increasing the pH of the protein causing amine groups in the protein to lose a hydrogen atom; and
contacting the amine groups with carboxylic acids in the polysaccharide, forming a covalent bond between the amine groups and the carboxylic acids; and
contacting the covalently bound protein and polysaccharide with the polyphenol, creating non-covalent interactions between the covalently bound protein and polysaccharide and the polyphenol.

17. A composition, comprising: curcumin, alginate and hemp protein, wherein:
the alginate and hemp protein are covalently bound to each other by a covalent bond between a carboxyl group on the alginate and an amine group on the hemp protein;
the curcumin is non-covalently complexed with the covalently bound alginate and hemp protein; and
the bioavailability of the curcumin in the composition is greater than the bioavailability of the curcumin in a composition lacking the covalently bound alginate and hemp protein.

18. The composition of claim 17, wherein the alginate is selected from propylene glycol alginate and fructooligosacharride.

19. The composition of claim 17 or claim 18, wherein the hemp protein is a globular hemp protein, or a peptide thereof.

20. The composition of claim 19, wherein the globular hemp protein, or peptide thereof, comprises one or more hydrophobic stretches of amino acid residues.

21. The composition of claim 20, wherein the one or more hydrophobic stretches of amino acid residues are the amino acid sequences of SEQ ID NO:1-SEQ ID NO:14.

22. A pharmaceutical composition, comprising the composition of any one of claims 17-21 and a pharmaceutically acceptable vehicle.

23. The pharmaceutical composition of claim 22, wherein the pharmaceutically acceptable vehicle is a capsule.

24. The pharmaceutical composition of claim 22, wherein the composition is a tablet.

25. A method of treating inflammation, comprising administering the pharmaceutical composition of any one of claims 22-24 to a subject with inflammation.

26. A method of improving cardiac endothelial function, comprising administering the pharmaceutical composition of any one of claims 22-24 to a subject with heart disease.

27. A method of treating osteoarthritis, comprising administering the pharmaceutical composition of any one of claims 22-24 to a subject with osteoarthritic pain.

28. A method of increasing Brain Derived Neurotrophic Factor (BDNF) and/or treating Alzheimer's Disease, comprising administering the pharmaceutical composition of any one of claims 22-24 to a subject in need thereof.

29. The method of any one of claims 25-28, wherein the treating, improving and/or increasing occurs by the curcumin impacting a biological pathway selected from mTOR, NFκb, NRF2 and autophagy/proteostasis.

30. A method of making the composition of any one of claims 17-21, comprising:
coating the alginate with the hemp protein via transacylation reactions by:
increasing the pH of the hemp protein causing amine groups in the hemp protein to lose a hydrogen atom; and
contacting the amine groups with carboxylic acids in the alginate, forming a covalent bond between the amine groups and the carboxylic acids; and
contacting the covalently bound alginate and hemp protein with the curcumin, creating non-covalent interactions between the covalently bound alginate and hemp protein and the curcumin.

31. A composition, comprising: berberine, propylene glycol alginate and pea protein, wherein:
the propylene glycol alginate and pea protein are covalently bound to each other by a covalent bond between a carboxyl group on the propylene glycol alginate and an amine group on the pea protein;
the berberine is non-covalently complexed with the covalently bound propylene glycol alginate and pea protein; and
the bioavailability of the berberine in the composition is greater than the bioavailability of the berberine in a composition lacking the covalently bound propylene glycol alginate and pea protein.

32. The composition of claim 31, wherein the pea protein is a globular pea protein, or a peptide thereof.

33. The composition of claim 32, wherein the globular pea protein, or peptide thereof, comprises one or more hydrophobic stretches of amino acid residues.

34. A pharmaceutical composition, comprising the composition of any one of claims 31-33 and a pharmaceutically acceptable vehicle.

35. The pharmaceutical composition of claim 34, wherein the pharmaceutically acceptable vehicle is a capsule.

36. The pharmaceutical composition of claim 34, wherein the composition is a tablet.

37. A method of treating diabetes, comprising administering the pharmaceutical composition of any one of claims 34-36 to a subject with diabetes.

38. The method of claim 37, wherein treating diabetes is accomplished by: reducing blood sugar, decreasing insulin resistance, increasing glycolysis, decreasing gluconeogenesis in the liver, slowing the breakdown of carbohydrates in the gut, or combinations thereof.

39. The method of claim 37 or claim 38, wherein the treating occurs by the berberine impacting a biological pathway selected from AMPK signaling pathway and c-myc.

40. A method of making the composition of any one of claims 31-33, comprising:
coating the propylene glycol alginate with the pea protein via transacylation reactions by:
increasing the pH of the pea protein causing amine groups in the pea protein to lose a hydrogen atom; and
contacting the amine groups with carboxylic acids in the propylene glycol alginate, forming a covalent bond between the amine groups and the carboxylic acids; and
contacting the covalently bound propylene glycol alginate and pea protein with the berberine, creating non-covalent interactions between the covalently bound propylene glycol alginate and pea protein and the berberine.

41. A composition, comprising: quercetin, propylene glycol alginate and pea protein, wherein:
the propylene glycol alginate and pea protein are covalently bound to each other by a covalent bond between a carboxyl group on the propylene glycol alginate and an amine group on the pea protein;
the quercetin is non-covalently complexed with the covalently bound propylene glycol alginate and pea protein; and
the bioavailability of the quercetin in the composition is greater than the bioavailability of the quercetin in a composition lacking the covalently bound propylene glycol alginate and pea protein.

42. The composition of claim 41, wherein the pea protein is a globular pea protein, or a peptide thereof.

43. The composition of claim 42, wherein the globular pea protein, or peptide thereof, comprises one or more hydrophobic stretches of amino acid residues.

44. A pharmaceutical composition, comprising the composition of any one of claims 41-43 and a pharmaceutically acceptable vehicle.

45. The pharmaceutical composition of claim 44, wherein the pharmaceutically acceptable vehicle is a capsule.

46. The pharmaceutical composition of claim 44, wherein the composition is a tablet.

47. A method of treating inflammation, comprising administering the pharmaceutical composition of any one of claims 44-46 to a subject with inflammation.

48. A method of treating allergy symptoms, comprising administering the pharmaceutical composition of any one of claims 44-46 to a subject with allergies.

49. A method of improving brain health by slowing the onset of Alzheimer's disease and/or dementia, comprising administering the pharmaceutical composition of any one of claims 44-46 to a subject in need thereof.

50. A method of lowering blood pressure, comprising administering the pharmaceutical composition of any one of claims 44-46 to a subject in need thereof.

51. The method of any one of claims 47-50, wherein the treating occurs by the quercetin impacting a biological pathway selected from mTOR, AMPK, and Senolytics.

52. A method of making the composition of any one of claims 41-43, comprising:
coating the propylene glycol alginate with the pea protein via transacylation reactions by:
increasing the pH of the pea protein causing amine groups in the pea protein to lose a hydrogen atom; and
contacting the amine groups with carboxylic acids in the propylene glycol alginate, forming a covalent bond between the amine groups and the carboxylic acids; and
contacting the covalently bound propylene glycol alginate and pea protein with the quercetin, creating non-covalent interactions between the covalently bound propylene glycol alginate and pea protein and the quercetin.

52. A composition, comprising: resveratrol, propylene glycol alginate and hemp protein, wherein:
the propylene glycol alginate and hemp protein are covalently bound to each other by a covalent bond between a carboxyl group on the propylene glycol alginate and an amine group on the hemp protein;
the resveratrol is non-covalently complexed with the covalently bound propylene glycol alginate and hemp protein; and
the bioavailability of the resveratrol in the composition is greater than the bioavailability of the resveratrol in a composition lacking the covalently bound propylene glycol alginate and hemp protein.

53. The composition of claim 52, wherein the hemp protein is a globular pea protein, or a peptide thereof.

54. The composition of claim 53, wherein the globular hemp protein, or peptide thereof, comprises one or more hydrophobic stretches of amino acid residues.

55. A pharmaceutical composition, comprising the composition of any one of claims 52-54 and a pharmaceutically acceptable vehicle.

56. The pharmaceutical composition of claim 55, wherein the pharmaceutically acceptable vehicle is a capsule.

57. The pharmaceutical composition of claim 55, wherein the composition is a tablet.

58. A method of treating inflammation, comprising administering the pharmaceutical composition of any one of claims 55-57 to a subject with inflammation.

59. A method of treating allergy symptoms, comprising administering the pharmaceutical composition of any one of claims 55-57 to a subject with allergies.

60. A method of improving brain health by slowing the onset of Alzheimer's disease and/or dementia, comprising administering the pharmaceutical composition of any one of claims 44-46 to a subject in need thereof.

61. A method of lowering blood pressure, comprising administering the pharmaceutical composition of any one of claims 55-57 to a subject in need thereof.

62. The method of any one of claims 58-61, wherein the treating occurs by the resveratrol impacting a biological pathway selected from mTOR, SIRT1/PGC1alpha, autophagy, and proteostasis.

63. A method of making the composition of any one of claims 52-54, comprising:
coating the propylene glycol alginate with the hemp protein via transacylation reactions by:
increasing the pH of the hemp protein causing amine groups in the hemp protein to lose a hydrogen atom; and
contacting the amine groups with carboxylic acids in the propylene glycol alginate, forming a covalent bond between the amine groups and the carboxylic acids; and
contacting the covalently bound propylene glycol alginate and hemp protein with the resveratrol, creating non-covalent interactions between the covalently bound propylene glycol alginate and hemp protein and the resveratrol.
